(19) United States
(12) Patent Application Publication
(10) Pub. No.: US 2010/0010037 A1
(43) Pub. Date: Jan. 14, 2010

(54) 1H-PYRROLO[2,3-B]PYRIDINE DERIVATIVES USEFUL AS HSP90 INHIBITORS

(75) Inventors: Paul Brough, Winnersh (GB); Martin Drysdale, Winnersh (GB)

Correspondence Address: BANNER & WITCOFF, LTD., 1100 13th STREET, N.W., SUITE 1200, WASHINGTON, DC, 20005-4051, US

(73) Assignee: VERNALIS (R&D) LTD., Winnersh (GB)

(21) Appl. No.: 12/438,809

(22) PCT Filed: Aug. 17, 2007
(86) PCT No.: PCT/GB2007/003133

§ 371 Date: Jun. 15, 2009

(30) Foreign Application Priority Data
Aug. 31, 2006 (GB) 0617161.5

(51) Int. Cl. A61K 31/437; C07D 471/02; A61P 35/04

(52) U.S. Cl. 514/300; 546/113

(57) ABSTRACT

Compounds of formula (I) have HSP90 inhibitory activity:

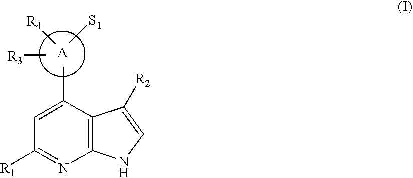

ring A is an aryl or heteroaryl ring or ring system; R₁ is hydrogen, fluoro, chloro, bromo, or a radical of formula (1A): —X-Alk¹-(Z)ₘ-(Alk²)ₙ-Q (IA) wherein X is a bond, —O—, —S—, —S(O)—, —SO₂—, or —NH—, Z is —O—, —S—, —(C═O)—, —(C═S)—, —S(O)—, —SO₂—, —NR^{A}—, or, in either orientation —C(═O)O—, —C(═O)NR^{A}—, —C(═S)NR^{A}—, —SO₂NR^{A}—, —NR^{A}C(═O)—, or —NR^{A}SO₂— wherein R^{A} is hydrogen or C₁-C₆ alkyl in which one or more hydrogens is optionally substituted by fluorine; Alk¹ and Alk² are optionally substituted divalent C₁-C₃ alkylene or C₂-C₃alkenylene radicals, m and n are independently 0 or 1, and Q is hydrogen or an optionally substituted carbocyclic or heterocyclic radical; R₂ is cyano (—CN), fluoro, chloro, bromo, methyl, ethyl, —OH, —CH₂OH, —C(═O)NH₂, —C(═O)H, —C(═O)CH₃, or —NH₂; R₃ and R₄ are independently selected from hydrogen, fluoro, chloro, bromo, cyano (—CN), C₁-C₃alkyl optionally substituted with one or more fluorine substituents, C₁-C₃alkoxy optionally substituted with one or more fluorine substituents, —CH═CH₂, —C≡CH, cyclopropyl and —NH₂, or R₃ and R₄ together represent methylenedioxy (—OCH₂O—) or ethylenedioxy (—OCH₂CH₂O—) in either of which one or more hydrogens are optionally replaced by fluorine; S₁ is as defined in the description.

This invention relates to aryl- or heteroaryl-substituted aza-indole (ie aryl- or heteroaryl-substituted 1H-pyrrolo[2,3-b]pyridine) compounds having HSP90 inhibitory activity, to the use of such compounds in medicine, in relation to diseases which are responsive to inhibition of HSP90 activity such as cancers, and to pharmaceutical compositions containing such compounds.

### BACKGROUND TO THE INVENTION

Molecular chaperones maintain the appropriate folding and conformation of proteins and are crucial in regulating the balance between protein synthesis and degradation. They have been shown to be important in regulating many important cellular functions, such as cell proliferation and apoptosis (Jolly and Morimoto, 2000; Smith et al., 1998; Smith, 2001).

### Heat Shock Proteins (Hsps)

Exposure of cells to a number of environmental stresses, including heat shock, alcohols, heavy metals and oxidative stress, results in the cellular accumulation of a number of chaperones, commonly known as heat shock proteins (Hsps). Induction of Hsps protects the cell against the initial stress insult, enhances recovery and leads to maintenance of a stress tolerant state. It has also become clear, however, that certain Hsps may also play a major molecular chaperone role under normal, stress-free conditions by regulating the correct folding, degradation, localization and function of a growing list of important cellular proteins.

A number of multigene families of Hsps exist, with individual gene products varying in cellular expression, function and localization. They are classified according to molecular weight, e.g., Hsp70, Hsp90, and Hsp27. Several diseases in humans can be acquired as a result of protein misfolding (reviewed in Tytell et al., 2001; Smith et al., 1998). Hence the development of therapies which disrupt the molecular chaperone machinery may prove to be beneficial. In some conditions (e.g., Alzheimer's disease, prion diseases and Huntington's disease), misfolded proteins can cause protein aggregation resulting in neurodegenerative disorders. Also, misfolded proteins may result in loss of wild type protein function, leading to deregulated molecular and physiological functions in the cell.

Hsps have also been implicated in cancer. For example, there is evidence of differential expression of Hsps which may relate to the stage of tumour progression (Martin et al., 2000; Conroy et al., 1996; Kawanishi et al., 1999; Jameel et al., 1992; Hoang et al., 2000; Lebeau et al., 1991). As a result of the involvement of Hsp90 in various critical oncogenic pathways and the discovery that certain natural products with anticancer activity are targeting this molecular chaperone suggests that inhibiting the function of Hsp90 may be useful in the treatment of cancer. To this end, the first in class natural product 17AAG is currently in Phase II clinical trials.

### Hsp90

Hsp90 constitutes about 1-2% of total cellular protein. In cells, it forms dynamic multi-protein complexes with a wide variety of accessory proteins (referred to as co-chaperones) which appear responsible for regulating the chaperone function. It is essential for cell viability and it exhibits dual chaperone functions (Young et al., 2001). When cells undergo various environmental cellular stresses, Hsp90 forms a core component of the cellular stress response by interacting with many proteins after their native conformation has been altered. Environmental stresses, such as heat shock, heavy metals or alcohol, generate localised protein unfolding. Hsp90 (in concert with other chaperones) binds these unfolded proteins allowing adequate refolding and preventing non-specific aggregation (Smith et al., 1998). In addition, recent results suggest that Hsp90 may also play a role in buffering against the effects of mutation, presumably by correcting the inappropriate folding of mutant proteins (Rutherford and Lindquist, 1998). However, Hsp90 also has an important regulatory role. Under normal physiological conditions, together with its endoplasmic reticulum homologue GRP94, Hsp90 plays a housekeeping role in the cell, maintaining the conformational stability and maturation of many client proteins. These can be subdivided into three groups: (a) steroid hormone receptors (e.g. estrogen receptor, progesterone receptor) (b) Ser/Thr or tyrosine kinases (e.g. Her2, Raf-1, CDK4, and Lck), and (c) a collection of apparently unrelated proteins, e.g. mutant p53 and the catalytic subunit of telomerase hTERT. It has also been shown recently that Hsp90 is responsible for stabilising and activating mutated kinases where the wild type kinase is not an Hsp90 client (for an example see the B-Raf story published in da Rocha Dias et al., 2005). All of these proteins play key regulatory roles in many physiological and biochemical processes in the cell. New client proteins of Hsp90 are being constantly identified; see http://www.picard.ch/downloads/Hsp90interactors.pdf for the most up to date list.

The highly conserved Hsp90 family in humans consists of four genes, namely the cytosolic Hsp90α and Hsp90β isoforms (Hickey et al., 1989), GRP94 in the endoplasmic reticulum (Argon et al., 1999) and Hsp75/TRAP1 in the mitochondrial matrix (Felts et al., 2000). Apart from the differences in sub-cellular localisation, very little is known about the differences in function between Hsp90α/β, GRP94 and TRAP1. Initial reports suggesting that certain client proteins were chaperoned by a specific Hsp90 (e.g. Her2 by Grp94 alone) appear to have been erroneous.

Hsp90 participates in a series of complex interactions with a range of client and regulatory proteins (Smith, 2001). Although the precise molecular details remain to be elucidated, biochemical and X-ray crystallographic studies (Prodromou et al., 1997; Stebbins et al., 1997) carried out over the last few years have provided increasingly detailed insights into the chaperone function of Hsp90.

Following earlier controversy on this issue, it is now clear that Hsp90 is an ATP-dependent molecular chaperone (Prodromou et al, 1997), with dimerisation of the nucleotide binding domains being essential for ATP hydrolysis, which is in turn essential for chaperone function (Prodromou et al, 2000a). Binding of ATP results in the formation of a toroidal dimer structure in which the N terminal domains are brought into closer contact with each other resulting in a conformational switch known as the ‘clamp mechanism’ (Prodromou and Pearl, 2000b). This conformational switching is, in part, regulated by the various co-chaperones associated with Hsp90 (Siligardi et al., 2004).

### Known Hsp90 Inhibitors

The first class of Hsp90 inhibitors to be discovered was the benzoquinone ansamycin class, which includes the compounds herbimycin A and geldanamycin. They were shown to reverse the malignant phenotype of fibroblasts transformed by the v-Src oncogene (Uehara et al., 1985), and subsequently to exhibit potent antitumour activity in both in vitro (Schulte et al., 1998) and in vivo animal models (Supko et al., 1995).

Immunoprecipitation and affinity matrix studies have shown that the major mechanism of action of geldanamycin involves binding to Hsp90 (Whitesell et al., 1994; Schulte and Neckers, 1998). Moreover, X-ray crystallographic studies have shown that geldanamycin competes at the ATP binding site and inhibits the intrinsic ATPase activity of Hsp90 (Prodromou et al., 1997; Panaretou et al., 1998). This interruption of the chaperone cycle (through blockage of the ATP turnover) causes the loss of the co-chaperone p23 from the complex and the targeting of the client proteins for degradation via the ubiquitin proteasome pathway. 17-Allylamino, 17-demethoxygeldanamycin (17AAG) retains the property of Hsp90 inhibition resulting in dient protein depletion and antitumour activity in cell culture and xenograft models (Schulte et al, 1998; Kelland et al, 1999), but has significantly less hepatotoxicity than geldanamycin (Page et al, 1997). Of interest, 17AAG has been shown to be much more active on tumour cells than its affinity for purified Hsp90 would suggest. This has lead to the suggestion that tumour cells (but not non-tumorigenic cells) contain a high-affinity conformation of Hsp90 to which 17AAG binds more tightly, and confers tumour selectivity on Hsp90 inhibitors (Kamal et al., 2003). 17AAG is currently being evaluated in Phase II clinical trials.

Radicicol is a macrocyclic antibiotic shown to reverse the malignant phenotype of v-Src and v-Ha-Ras transformed fibroblasts (Kwon et al, 1992; Zhao et al, 1995). It was shown to degrade a number of signalling proteins as a consequence of Hsp90 inhibition (Schulte et al., 1998). X-ray crystallographic data confirmed that radicicol also binds to the N terminal domain of Hsp90 and inhibits the intrinsic ATPase activity (Roe et al., 1998). Radicicol lacks antitumour activity in vivo due to the unstable chemical nature of the compound.

Coumarin antibiotics are known to bind to bacterial DNA gyrase at an ATP binding site homologous to that of the Hsp90. The coumarin, novobiocin, was shown to bind to the carboxy terminus of Hsp90, i.e., at a different site to that occupied by the benzoquinone ansamycins and radicicol which bind at the N-terminus (Marcu et al., 2000b). However, this still resulted in inhibition of Hsp90 function and degradation of a number of Hsp90-chaperoned signalling proteins (Marcu et al., 2000a). Geldanamcyin cannot bind Hsp90 subsequent to novobiocin; this suggests that some interaction between the N and C terminal domains must exist and is consistent with the view that both sites are important for Hsp90 chaperone properties.

A purine-based Hsp90 inhibitor, PU3, has been shown to result in the degradation of signalling molecules, including Her2, and to cause cell cycle arrest and differentiation in breast cancer cells (Chiosis et al., 2001). Recent studies have identified other purine-based compounds with activity against Her2 and activity in cell growth inhibition assays (Dymock et al 2004; Kasibhatla et al 2003; Llauger et al 2005).

Patent publications WO 2004/050087, WO 2004/056782, WO 2004/072051, WO 2004/096212, WO 2005/000300, WO 2005/021552, WO 2005/034950, WO 2005/063222; WO 2005/028434; WO 2005/061461, WO2006/008503 WO 2006/0010594 WO 2006/0010595 WO 2006/0014744 WO 2006/018082 WO 2006/039977 WO 2006/051808 WO 2006/055760 WO 2006/075095 WO 2006/079789 WO 2006/084030 and WO 2006/087077 also relate to inhibition of Hsp90.

Hsp90 as a Therapeutic Target

Due to its involvement in regulating a number of signalling pathways that are crucially important in driving the phenotype of a tumour, and the discovery that certain bioactive natural products exert their effects via Hsp90 activity, the molecular chaperone Hsp90 is currently being assessed as a new target for anticancer drug development (Neckers et al., 1999).

The predominant mechanism of action of geldanamycin, 17AAG, and radicicol involves binding to Hsp90 at the ATP binding site located in the N-terminal domain of the protein, leading to inhibition of the intrinsic ATPase activity of Hsp90 (Prodromou et al., 1997; Stebbins et al., 1997; Panaretou et al., 1998).

Inhibition of Hsp90 ATPase activity by 17AAG induces the loss of p23 from the chaperone-client protein complex interrupting the chaperone cycle. This leads to the formation of a Hsp90-client protein complex that targets these client proteins for degradation via the ubiquitin proteasome pathway (Neckers et al., 1999; Whitesell & Lindquist, 2005). Treatment with Hsp90 inhibitors leads to selective degradation of important proteins (for example Her2, Akt, estrogen receptor and CDK4) involved in cell proliferation, cell cycle regulation and apoptosis, processes which are fundamentally important in cancer.

The preclinical development of 17AAG as an anticancer agent has been well documented (Sausville et al., 2003) and is currently undergoing Phase II clinical trials. Phase I clinical trials results have been recently published (Banerji et al., 2005; Goetz et al., 2005; Ramanathan et al., 2005 and Grem et al., 2005). Of all these trials, the one conducted by Banerji et al. proved the most positive with a maximum dose of 450 mg/m2/week achieved with PD marker responses in the majority of patients and possible antitumour activity in two patients

Inhibition of Hsp90 function has been shown to cause selective degradation of important signalling proteins involved in cell proliferation, cell cycle regulation and apoptosis, processes which are fundamentally important and which are commonly deregulated in cancer (Hostein et al., 2001). An attractive rationale for developing drugs against this target for use in the clinic is that by simultaneously depleting proteins associated with the transformed phenotype, one may obtain a strong antitumour effect and achieve a therapeutic advantage against cancer versus normal cells. These events downstream of Hsp90 inhibition are believed to be responsible for the antitumour activity of Hsp90 inhibitors in cell culture and animal models (Schulte et al., 1998; Kelland et al., 1999).

Recent work has shown that the acetylation status of Hsp90 also plays a role in the control of the chaperone cycle. Inhibition of HDAC6 by either small molecule inhibitors or through siRNA gene targeting interrupts the chaperone cycle. Such treatments cause client protein degradation in a fashion analogous to small molecule ATP site inhibitors (Kovacs et al, 2005; Aoyagi & Archer, 2005).

### BRIEF DESCRIPTION OF THE INVENTION

This invention is based on the finding that a class of aryl- or heteroaryl-substituted aza-indole compounds has Hsp90 inhibitory activity, and is of interest in the treatment of diseases responsive to inhibition of Hsp90 activity.

Patent publication WO 2006/046023 is concerned with ortho-condensed pyridine and pyrimidine derivatives (eg purines) as protein kinase inhibitors. The definition of the compounds with which that publication is concerned is very broad, but no aryl- or heteroaryl-substituted aza-indole compounds are particularized or exemplified therein. Also, since the publication is concerned with protein kinase inhibitors, it provides no information concerning the activity of 4-aryl or 4-heteroaryl aza-indole derivatives against Hsp90.

### DETAILED DESCRIPTION OF THE INVENTION

In one broad aspect the present invention provides a compound of formula (I), or a salt, N-oxide, hydrate, or solvate thereof:

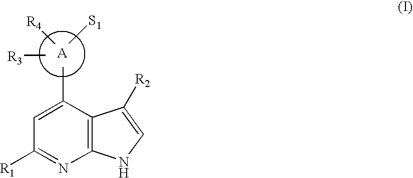

wherein

ring A is an aryl or heteroaryl ring or ring system;

R₁ is hydrogen, fluoro, chloro, bromo, or a radical of formula (1A):

—X-Alk¹-(Z)ₘ-(Alk²)ₙ-Q (IA)

wherein
X is a bond, —O—, —S— —S(O)—, —SO₂—, or —NH—,
Z is —O—, —S—, —(C═O)—, —(C═S)—, —S(O)—, —SO₂—, —NR^{A}—, or, in either orientation —C(═O)O—, —C(═O)NR^{A}—, —C(═S)NR^{A}—, —SO₂NR^{A}, —NR^{A}C(═O), or —NR^{A}SO₂— wherein R^{A} is hydrogen or C₁-C₆ alkyl in which one or more hydrogens is optionally substituted by fluorine;
Alk¹ and Alk² are optionally substituted divalent C₁-C₃ alkylene or C₂-C₃ alkenylene radicals,

m, and n are independently 0 or 1, and

Q is hydrogen or an optionally substituted carbocyclic or heterocyclic radical;

R₂ is cyano (—CN), fluoro, chloro, bromo, methyl, ethyl, —OH, —CH₂OH, —C(═O)NH₂, —C(═O)H, —C(═O)CH₃, or —NH₂;

R₃ and R₄ are independently selected from hydrogen, fluoro, chloro, bromo, cyano (—CN), C₁-C₃alkyl optionally substituted with one or more fluorine substituents, C₁-C₃alkoxy optionally substituted with one or more fluorine substituents, —CH═CH₂, —C≡CH, cyclopropyl and —NH₂, or R₃ and R₄ together represent methylenedioxy (—OCH₂O—) or ethylenedioxy (—OCH₂CH₂O—) in either of which one or more hydrogens are optionally replaced by fluorine;

S₁ is hydrogen, or a substituent selected from fluoro, chloro, bromo, cyano (—CN), C₁-C₃alkyl optionally substituted with one or more fluorine substituents, C₁-C₃alkoxy optionally substituted with one or more fluorine substituents, —CH═CH₂, —C≡CH, cyclopropyl and —NH₂, or S₁ and R₃, or S₁ and R₄, together represent methylenedioxy (—OCH₂O—) or ethylenedioxy ((—OCH₂ CH₂O—) in either of which one or more hydrogens are optionally replaced by fluorine; or S₁ is a radical of formula (IB):

-(Alk³)ₚ-(Z¹)_{q}-(Alk⁴)ᵣ-Q¹ (IB)

wherein
p, q and r are independently 0 or 1;
(a) when p is 0 or 1, and q is 1, and r is 0 or 1:
Z¹ is selected from the group of divalent radicals consisting of (i) —S—, —(C═O)—, —(C═S)—, —S(O)— and —SO₂— and (ii) —N(R^{A})C(═O)—* wherein the bond marked * is attached to Q¹ and (iii) in either orientation, —C(═O)O—, —C(═S)NR^{A}—, and —SO₂NR^{A}—; and Q¹ is (i) hydrogen or an optional substituent; or (ii) an optionally substituted carbocyclic or heterocyclic radical; or (iii) a radical —CH₂[O(CH₂)_{w}]ₓZ² wherein Z² is H, —OH or —O(C₁-C₃alkyl) wherein x and w are independently 1, 2 or 3; or
(b) when p is 1, and q is 1, and r is 0 or 1:
Z¹ is —O—, and Q¹ is (i) hydrogen or an optional substituent which is not linked to -(Alk³)ₚ-(Z¹)_{q}-(Alk⁴)ᵣ- through a nitrogen atom; or (ii) an optionally substituted carbocyclic radical; or (iii) an optionally substituted heterocyclic ring of 5 or 6 ring atoms which is not linked to -(Alk³)ₚ-(Z¹)_{q}-(Alk⁴)ᵣ- through a ring nitrogen; or (iv) a radical —CH₂[O(CH₂)_{w}]ₓZ² wherein Z² is H, —OH or —O(C₁-C₃alkyl) wherein x and w are independently 1, 2 or 3, or
(c) when p is 1, and q is 1, and r is 0 or 1:
Z¹ is —NR^{A}— or —C(═O)N(R^{A})—* wherein the bond marked * is attached to Q¹ and Q¹ is a radical —CH₂[O(CH₂)_{w}]ₓZ² wherein Z² is H, —OH or —O(C₁-C₃alkyl) wherein x and w are independently 1, 2 or 3, or
(d) when p is 0, and q is 1, and r is 0 or 1:
Z¹ is —O— or —NR^{A}— and Q′ is (i) hydrogen or an optional substituent which is not linked to -(Alk³)ₚ-(Z¹)_{q}-(Alk⁴)ᵣ- through a nitrogen atom; or (ii) Q¹ and R^{A}, taken together with the nitrogen to which they are attached form an optionally substituted heterocyclic ring of 5 or 6 ring atoms; or (iii) a radical —CH₂[O(CH₂)_{w}]ₓZ² wherein Z² is H, —OH or —O(C₁-C₃alkyl) wherein x and w are independently 1, 2 or 3; or
(e) when p is 0 or 1, q is 0, and r is 0 or 1:
Q¹ is (i) hydrogen or an optional substituent which is not linked to -(Alk³)ₚ-(Z¹)_{q}-(Alk⁴)ᵣ- through a nitrogen atom or (ii) an optionally substituted carbocyclic radical; or (iii) an optionally substituted heterocyclic of 5 or 6 ring atoms which is not linked to -(Alk³)ₚ-(Z¹)_{q}-(Alk⁴)ᵣ- through a ring nitrogen; or (iv) a radical —CH₂[O(CH₂)_{w}]ₓZ² wherein Z² is H, —OH or —O(C₁-C₃alkyl) wherein x and w are independently 1, 2 or 3;
R^{A} is hydrogen or C₁-C₃ alkyl optionally substituted with one or more fluorine substituents; and
Alk³ and Alk⁴ are divalent C₁-C₃ alkylene or C₂-C₃ alkenylene radicals, each optionally substituted by one or two substituents selected from fluoro, chloro, C₁-C₃alkyl optionally substituted with one or more fluorine substituents, C₁-C₃alkoxy optionally substituted with one or more fluorine substituents.

In another broad aspect, the invention provides a compound of formula (IC), or a salt, N-oxide, hydrate, or solvate thereof:

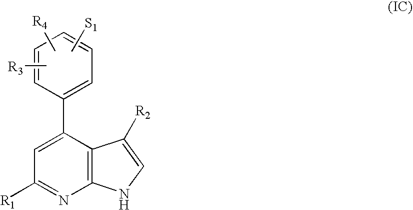

wherein

R₁, R₂, R₃ and R₄ are as defined in relation to formula (I) above, and

S₁ is hydrogen, or a substituent selected from fluoro, chloro, bromo, cyano (—CN), C₁-C₃alkyl optionally substituted with one or more fluorine substituents, C₁-C₃alkoxy optionally substituted with one or more fluorine substituents, —CH═CH₂, —C≡CH, cyclopropyl and —NH₂, or S₁ and R₃, or S₁ and R₄, together represent methylenedioxy (—OCH₂O—) or ethylenedioxy ((—OCH₂ CH₂O—) in either of which one or more hydrogens are optionally replaced by fluorine; or SI is a radical of formula (IB):

-(Alk₃)ₚ-(Z¹)_{q}-(Alk⁴)ᵣ-Q¹ (IB)

wherein
p, q and r are independently 0 or 1;
Z¹ is —O—, —S—, —(C═O)—, —(C═S)—, —S(O)—, —SO₂—, —NR^{A}—, or, in either orientation, —C(═O)N(R^{A})— or —SO₂NR^{A}—;
Q¹ is (i) hydrogen or an optional substituent; or (ii) an optionally substituted carbocyclic or heterocyclic radical; or (iii) a radical —CH₂[O(CH₂)_{w}]ₓZ² wherein Z² is H, —OH or —O(C₁-C₃alkyl) wherein x and w are independently 1, 2 or 3;
R^{A} is hydrogen or C₁-C₃ alkyl optionally substituted with one or more fluorine substituents; and
Alk³ and Alk⁴ are divalent C₁-C₃ alkylene or C₂-C₃ alkenylene radicals, each optionally substituted by one or two substituents selected from fluoro, chloro, C₁-C₃alkyl optionally substituted with one or more fluorine substituents, C₁-C₃alkoxy optionally substituted with one or more fluorine substituents.

In another aspect, the invention provides the use of a compound of formula (I) or (IC), or a salt, N-oxide, hydrate, or solvate thereof in the preparation of a composition for inhibition of HSP90 activity in vitro or in vivo:

The invention also provides a method of treatment of diseases which are responsive to inhibition of HSP90 activity in mammals, which method comprises administering to the mammal an amount of a compound as defined above effective to inhibit said HSP90 activity.

The in vivo use, and method, of the invention is applicable to the treatment of diseases in which HSP90 activity is implicated, including use for immunosuppression or the treatment of viral disease, inflammatory diseases such as rheumatoid arthritis, asthma, multiple sclerosis, Type I diabetes, lupus, psoriasis and inflammatory bowel disease; cystic fibrosis angiogenesis-related disease such as diabetic retinopathy, haemangiomas, and endometriosis; or for protection of normal cells against chemotherapy-induced toxicity; or diseases where failure to undergo apoptosis is an underlying factor; or protection from hypoxia-ischemic injury due to elevation of Hsp70 in the heart and brain; scrapie/CJD, Huntingdon's or Alzheimer's disease. Use for the treatment of cancer is especially indicated.

As used herein, the term “(Cₐ-C_{b})alkyl” wherein a and b are integers refers to a straight or branched chain alkyl radical having from a to b carbon atoms. Thus when a is 1 and b is 6, for example, the term includes methyl, ethyl, n-propyl, isopropyl, n-butyl, isobutyl, sec-butyl, t-butyl, n-pentyl and n-hexyl.

As used herein the term “divalent (Cₐ-C_{b})alkylene radical” wherein a and b are integers refers to a saturated hydrocarbon chain having from a to b carbon atoms and two unsatisfied valences.

As used herein the term “(Cₐ-C_{b})alkenyl” wherein a and b are integers refers to a straight or branched chain alkenyl moiety having from a to b carbon atoms having at least one double bond of either E or Z stereochemistry where applicable. The term includes, for example, vinyl, allyl, 1- and 2-butenyl and 2-methyl-2-propenyl.

As used herein the term “divalent (Cₐ-C_{b})alkenylene radical” refers to a hydrocarbon chain having from a to b carbon atoms, at least one double bond, and two unsatisfied valences.

As used herein the term “cycloalkyl” refers to a saturated carbocyclic radical having from 3-8 carbon atoms and includes, for example, cyclopropyl, cyclobutyl, cyclopentyl, cyclohexyl, cycloheptyl and cyclooctyl.

As used herein the term “cycloalkenyl” refers to a carbocyclic radical having from 3-8 carbon-atoms containing at least one double bond, and includes, for example, cyclopentenyl, cyclohexenyl, cycloheptenyl and cyclooctenyl.

As used herein the term “aryl” refers to a mono-, bi- or tri-cyclic carbocyclic aromatic radical, and includes aromatic monocyclic or bicyclic carbocyclic radicals fused to a non aromatic carbocyclic or heterocyclic ring. Illustrative of such radicals are phenyl, biphenyl and napthyl, and radicals of the formula:

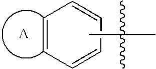

wherein ring A (i) is optionally substituted, (ii) has 5 or 6 ring members including the carbons of the phenyl ring to which it is fused, and (iii) has at least one heteroatom O, S or N hetero atom as a ring member.

As used herein the term “carbocyclic” refers to a cyclic radical whose ring atoms are all carbon, and includes aryl, cycloalkyl, and cycloalkenyl radicals.

As used herein the term “heteroaryl” refers to a mono-, bi- or tri-cyclic aromatic radical containing one or more heteroatoms selected from S, N and O. Illustrative of such radicals are thienyl, benzthienyl, furyl, benzfuryl, pyrrolyl, imidazolyl, benzimidazolyl, thiazolyl, benzthiazolyl, isothiazolyl, benzisothiazolyl, pyrazolyl, oxazolyl, benzoxazolyl, isoxazolyl, benzisoxazolyl, isothiazolyl, triazolyl, benztriazolyl, thiadiazolyl, oxadiazolyl, pyridinyl, pyridazinyl, pyrimidinyl, pyrazinyl, triazinyl, indolyl and indazolyl.

As used herein the unqualified term “heterocyclyl” or “heterocyclic” includes “heteroaryl” as defined above, and in particular refers to a mono-, bi- or tri-cyclic non-aromatic radical containing one or more heteroatoms selected from S, N and O, and to groups consisting of a monocyclic non-aromatic radical containing one or more such heteroatoms which is covalently linked to another such radical or to a monocyclic carbocyclic radical. Illustrative of such radicals are pyrrolyl, furanyl, thienyl, piperidinyl, imidazolyl, oxazolyl, isoxazolyl, thiazolyl, thiadiazolyl, pyrazolyl, pyridinyl, pyrrolidinyl, pyrimidinyl, morpholinyl, piperazinyl, indolyl, morpholinyl, benzofuranyl, pyranyl, isoxazolyl, benzimidazolyl, methylenedioxyphenyl, ethylenedioxyphenyl, maleimido and succinimido groups.

Unless otherwise specified in the context in which it occurs, the term “substituted” as applied to any moiety herein means substituted with at least one substituent, for example selected from (C₁-C₆)alkyl, (C₁-C₆)alkoxy, hydroxy, hydroxy(C₁-C₆)alkyl, mercapto, mercapto(C₁-C₆)alkyl, (C₁-C₆)alkylthio, monocyclic carbocyclic of 3-6 ring carbon atoms, monocyclic heterocyclic of 5 or 6 ring atoms, halo (including fluoro and chloro), trifluoromethyl, trifluoromethoxy, nitro, nitrile (—CN), oxo, —COOH, —COOR^{A}, —COR^{A}, —SO₂R^{A}, —CONH₂, —SO₂NH₂, —CONHR^{A}, —SO₂NHR^{A}, —CONR^{A}R^{B}, —SO₂NR^{A}R^{B}, —NH₂, —NHR^{A}, —NR^{A}R^{B}, —OCONH₂, —OCONHR^{A}, —OCONR^{A}R^{B}, —NHCOR^{A}, —NHCOOR^{A}, —NR^{B}COOR^{A}, —NHSO₂OR^{A}, —NR^{B}SO₂OR^{A}, —NHCONH₂, —NR^{A}CONH₂, —NHCONHR^{B}, NR^{A}CONHR^{B}, —NHCONR^{A}R^{B}, or —NR^{A}CONR^{A}R^{B} wherein R^{A} and R^{B} are independently a (C₁-C₆)alkyl group in which one or more nitrogens are optionally replaced by fluorine. In the case where the optional substituent contains an alkyl radical, that alkyl radical may be substituted by one or more fluorines, and/or by a monocyclic carbocyclic group of 3-6 ring carbon atoms, or a monocyclic heterocyclic group of 5 or 6 ring atoms. In the case where the optional substituent is or comprises a monocyclic carbocyclic group of 3-6 ring carbon atoms, or a monocyclic heterocyclic group of 5 or 6 ring atoms, that ring may itself be substituted by any of the non-cyclic optional substituents listed above. An “optional substituent” may be one of the substituent groups encompassed in the above description.

As used herein the term “salt” includes base addition, acid addition and quaternary salts. Compounds of the invention which are acidic can form salts, including pharmaceutically or veterinarily acceptable salts, with bases such as alkali metal hydroxides, e.g. sodium and potassium hydroxides; alkaline earth metal hydroxides e.g. calcium, barium and magnesium hydroxides; with organic bases e.g. N-ethyl piperidine, dibenzylamine and the like. Those compounds (I) which are basic can form salts, including pharmaceutically or veterinarily acceptable salts with inorganic acids, e.g. with hydrohalic acids such as hydrochloric or hydrobromic acids, sulphuric acid, nitric acid or phosphoric acid and the like, and with organic acids e.g. with acetic, tartaric, succinic, fumaric, maleic, malic, salicylic, citric, methanesulphonic and p-toluene sulphonic acids and the like.

For a review on suitable salts, see Handbook of Pharmaceutical Salts: Properties, Selection, and Use by Stahl and Wermuth (Wiley-VCH, Weinheim, Germany, 2002).

The term ‘solvate’ is used herein to describe a molecular complex comprising the compound of the invention and a stoichiometric amount of one or more pharmaceutically acceptable solvent molecules, for example, ethanol. The term ‘hydrate’ is employed when said solvent is water.

Compounds with which the invention is concerned which may exist in one or more stereoisomeric form, because of the presence of asymmetric atoms or rotational restrictions, can exist as a number of stereoisomers with R or S stereochemistry at each chiral centre or as atropisomers with R or S stereochemistry at each chiral axis. The invention includes all such enantiomers and diastereoisomers and mixtures thereof.

So-called ‘pro-drugs’ of the compounds of formula (I) or (IC) are also within the scope of the invention. Thus certain derivatives of compounds of formula (I) or (IC) which may have little or no pharmacological activity themselves can, when administered into or onto the body, be converted into compounds of formula (I) or (IC) having the desired activity, for example, by hydrolytic cleavage. Such derivatives are referred to as ‘prodrugs’. Further information on the use of prodrugs may be found in Pro-drugs as Novel Delivery Systems, Vol. 14, ACS Symposium Series (T. Higuchi and W. Stella) and Bioreversible Carriers in Drug Design, Pergamon Press, 1987 (ed. E. B. Roche, American Pharmaceutical Association).

Prodrugs in accordance with the invention can, for example, be produced by replacing appropriate functionalities present in the compounds of formula (I) or (IC) with certain moieties known to those skilled in the art as ‘pro-moieties’ as described, for example, in Design of Prodrugs by H. Bundgaard (Elsevier, 1985).

Also included within the scope of the invention are metabolites of compounds of formula (I) or (IC), that is, compounds formed in vivo upon administration of the drug. Some examples of metabolites include
(i) where the compound of formula (I) or (IC) contains a methyl group, an hydroxymethyl derivative thereof (—CH₃—>—CH₂OH):
(ii) where the compound of formula (I) or (IC) contains an alkoxy group, an hydroxy derivative thereof (—OR—>—OH);
(iii) where the compound of formula (I) or (IC) contains a tertiary amino group, a secondary amino derivative thereof (—NR¹R²—>—NHR¹ or —NHR²);
(iv) where the compound of formula (I) or (IC) contains a secondary amino group, a primary derivative thereof (—NHR—>—NH₂);
(v) where the compound of formula (I) or (IC) contains a phenyl moiety, a phenol derivative thereof (-Ph->-PhOH); and
(vi) where the compound of formula (I) or (IC) contains an amide group, a carboxylic acid derivative thereof (—CONH₂—>COOH).

The Group R₁ in Compounds (I) and (IC)

When R₁ is a radical of formula (IA):

—X-Alk¹-(Z)ₘ-(Alk²)ₙ-Q (IA)
X may be —O—, —S— —S(O)—, —SO₂—, or —NH—. At present —O— and —S— are preferred;
when present, Z may be —O—, —S—, —(C═O)—, —(C═S)—, —S(O)—, —SO₂—, —NR^{A}—, or, in either orientation —C(═O)O—, —C(═O)NR^{A}—, —C(═S)NR^{A}—, —SO₂NR^{A}—, —NR^{A}C(═O)—, or —NR^{A}SO₂— wherein R^{A} is hydrogen or C₁-C₆ alkyl. At present —NR^{A}— is preferred;
Alk¹ (and Alk² when present) may be, for example —CH₂—, —CH₂CH₂—, —CH₂CH₂CH₂—, —CH(CH₃)CH₂— or —CH₂CH═CH—;
m and n are independently 0 or 1. Thus, in one class of radicals (IA), m and n are both 0. In another class of radicals (IA), m is 1 and n is 0. In a further class of radicals (IA), m is 0 and n is 1;
Q may be hydrogen or an optionally substituted carbocyclic or heterocyclic radical. Examples of carbocyclic radicals Q include phenyl, cyclopropyl, cyclobutyl, cyclopentyl and cyclohexyl. Examples of heterocyclic radicals Q include heteroaryl radicals such as pyridyl, thienyl and furanyl, and non-aromatic heterocyclic radicals such as piperidinyl, piperazinyl and morpholinyl.
Currently it is preferred that Alk¹, Alk² and Q (when carbocyclic or heterocyclic) are unsubstituted. However, examples of substituents which may be present in Alk¹, Alk₂ and Q (when carbocyclic or heterocyclic) include methyl, ethyl, n- or isopropyl, vinyl, allyl, methoxy, ethoxy, n-propyloxy, isopropyloxy, benzyloxy, allyloxy, cyanomethoxy chloro, bromo, cyano, formyl, methyl-, ethyl-, or n-propyl-carbonyloxy, methyl- or ethylaminocarbonyl, and substituents of formula —O(CH₂)ₐZ¹ wherein a is 1, 2 or 3 and Z¹ is a primary, secondary, tertiary or cyclic amino group, or a C₁-C₆alkoxy group, and substituents of formula -(Alk³)_{b}Z¹ wherein Alk³ is a divalent straight or branched chain (C₁-C₃) alkylene, b is 0 or 1, and Z¹ is a primary, secondary, tertiary or cyclic amino group, or a C₁-C₆alkoxy group.

In one class of compounds (I) of the invention R₁ is methoxy, ethoxy, methylthio or ethylthio.

The Group R₂ in Compounds (I) and (IC)

At present, it is preferred that R₂ is cyano (—CN).

The ring A in Compounds (I)

Ring A is an aryl or heteroaryl ring or ring system, for example phenyl, thienyl, benzothienyl, furyl, benzofuryl, pyrrolyl, imidazolyl, benzimidazolyl, thiazolyl, benzthiazolyl, isothiazolyl, benzisothiazolyl, pyrazolyl, oxazolyl, benzoxazolyl, isoxazolyl, benzisoxazolyl, isothiazolyl, triazolyl, benztriazolyl, thiadiazolyl, oxadiazolyl, pyridinyl, pyridazinyl, pyrimidinyl, pyrazinyl, triazinyl, indolyl or indazolyl. Currently it is preferred that ring A is phenyl.

The Groups R₃ and R₄ in Compounds (I) and (IC)

R₃ and R₄ are independently selected from hydrogen, fluoro, chloro, bromo, cyano (—CN), C₁-C₃alkyl optionally substituted with one or more fluorine substituents, C₁-C₃alkoxy optionally substituted with one or more fluorine substituents, —CH═CH₂, —C═CH, cyclopropyl and —NH₂, or R₃ and R₄ together represent methylenedioxy (—OCH₂O—) or ethylenedioxy (—OCH₂CH₂O—) in either of which one or more hydrogens are optionally replaced by fluorine. However at least one of R₃ and R₄ should preferably be other than hydrogen. Presently preferred is the case where one or both of R₃ and R₄ is/are selected from fluoro, chloro, methyl or methoxy. Preferred positions for R₃ and R₄ when ring A is phenyl are the para and ortho positions.

The Group S₁ in Compounds (I)

When S₁ is other than hydrogen, and ring A is phenyl, it is presently preferred that S₁ be in the meta position of the ring.

In a first subset of compounds (I) of the invention, S₁ is hydrogen, or a substituent selected from fluoro, chloro, bromo, cyano (—CN), C₁-C₃alkyl optionally substituted with one or more fluorine substituents, C₁-C₃alkoxy optionally substituted with one or more fluorine substituents, —CH═CH₂, —C≡CH, cyclopropyl and —NH₂, or S₁ and R₃, or S₁ and R₄, together represent methylenedioxy (—OCH₂O—) or ethylenedioxy (—OCH₂CH₂O—) in either of which one or more hydrogens are optionally replaced by fluorine;

In a second subset of compounds (I) of the invention, S₁ is a radical of formula (IB):

-(Alk³)ₚ-(Z¹)_{q}-(Alk⁴)ᵣ-Q¹ (IB)

wherein p, q, r, Alk³, Alk⁴, Z¹ and Q¹ are as defined in relation to formula (I) above. In such compounds, Alk³ and Alk⁴ are divalent C₁-C₃ alkylene or C₂-C₃ alkenylene radicals, each optionally substituted by one or two substituents selected from fluoro, chloro, C₁-C₃alkyl optionally substituted with one or more fluorine substituents, C₁-C₃alkoxy optionally substituted with one or more fluorine substituents. Examples of radicals Alk³ and Alk⁴, when present, are example —CH₂—, —CH₂CH₂—, CH₂CH₂CH₂—, CH(CH₃)CH₂—, —CH₂CH(CH₃)CH₂—, —CH₂CH═CH—; —CH(OCH₃)CH₂—, and —CH₂CH(OCH₃)CH₂—,

In this second subset of compounds (I) of the invention there are five specific combinations (a)-(e) of p, q, r, Alk³, Alk⁴, Z¹ and Q¹:

Case (a) arises when p is 0 or 1, and q is 1, and r is 0 or 1. In case (a), Z¹ is selected from the group of divalent radicals consisting of (i) —S—, —(C═O)—, —(C═S)—, —S(O)— and —SO₂— and (ii) —N(R^{A})C(═O)—* wherein the bond marked * is attached to Q¹ and (iii) in either orientation, —C(═O)O—, —C(═S)NR^{A}—, and —SO₂NR^{A}—; and Q¹ is (i) hydrogen or an optional substituent; or (ii) an optionally substituted carbocyclic or heterocyclic radical; or (iii) a radical —CH₂[O(CH₂)_{w}]ₓZ² wherein Z² is H, —OH or —O(C₁-C₃alkyl) wherein x and w are independently 1, 2 or 3.

R^{A}, when present in Z¹ and when other than hydrogen may be, for example, methyl, ethyl, n- or iso-propyl, or trifluoromethyl.

When other than hydrogen, Q¹ may be, for example
an primary, secondary or tertiary amino substituent, for example —NR^{A}R^{B} wherein R^{A} and R^{B} are independently selected from hydrogen and C¹-C₃alkyl in which one or more hydrogens is optionally replaced by fluorine, for example methylamino, dimethylamino, ethylamino, diethylamino, n- or iso-propylamino, or N-methyl-N-ethylamino and N-(1,1,1-trifluoroethyl)-N-ethylamino,
a non-amino optional substituent, for example chloro, C₁-C₃alkoxy, cyano or acetyl; or a cyclopropyl, cylopenyl or cyclohexyl group;
an optionally substituted phenyl group, for example wherein optional substituents are selected from cyano (—CN), fluoro, chloro, bromo, methyl, ethyl, —OH, —CH₂OH, —C(═O)NH₂, —C(═O)H, —C(═O)CH₃, and —NH₂;
a cyclic amino group such as morpholino, piperidinyl, piperazinyl or methylpiperidinyl or a fluoro substituted cyclic amino group such as those of formulae (A)-(D):

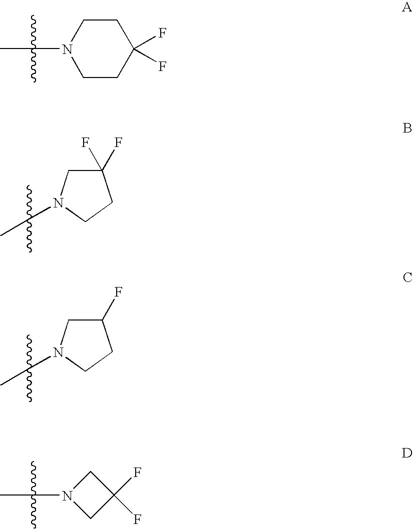

a saturated carbocylic group such as cyclopropyl, cyclopentyl, cyclohexyl or norbornyl;
a heterocyclic group such as any of those heteroaryl groups referred to above as examples of ring A, or a non aromatic heterocyclic group such as one having the formula E:

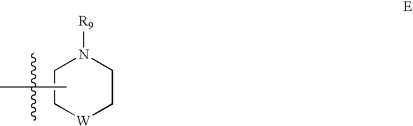

wherein W is —CH₂—, —O—, —S— or —NR₉, and R₉ is hydrogen, methyl, ethyl or n- or iso-propyl; or
a radical —CH₂[O(CH₂)_{w}]ₓZ² wherein Z² is H, —OH or —OCH₃ wherein x and w are independently 1, 2 or 3. Such radicals include the polyether radicals —O—(CH₂)₁₋₃OH, —O—(CH₂)₁₋₃O(C₁-C₃alkyl), —O—(CH₂)₁₋₃—O—(CH₂)₁₋₃OH, and —O—(CH₂)₁₋₃—O—(CH₂)₁₋₃O(C₁-C₃alkyl),

Case (b) arises when p is 1, and q is 1, and r is 0 or 1 and Z¹ is —O—. In case (b) Q¹ is (i) hydrogen or an optional substituent which is not linked to -(Alk³)ₚ-(Z)_{q}-(Alk⁴)ᵣ- through a nitrogen atom; or (ii) an optionally substituted carbocyclic radical; or (iii) an optionally substituted heterocyclic ring of 5 or 6 ring atoms which is not linked to -(Alk³)ₚ-(Z¹)_{q}-(Alk⁴)ᵣ- through a ring nitrogen; or (iv) a radical —CH₂[O(CH₂)_{w}]ₓZ² wherein Z² is H, —OH or —O(C₁-C₃alkyl) wherein x and w are independently 1, 2 or 3.

In this case (b), when other than hydrogen, Q¹ may be, for example
a non-amino optional substituent, for example chloro, C₁-C₃alkoxy, cyano or acetyl; or a cyclopropyl, cylopenyl or cyclohexyl group;
an optionally substituted phenyl group, for example wherein optional substituents are selected from cyano (—CN), fluoro, chloro, bromo, methyl, ethyl, —OH, —CH₂OH, —C(═O)NH₂, —C(═O)H, —C(═O)CH₃, and —NH₂;
a saturated carbocylic group such as cyclopropyl, cyclopentyl, cyclohexyl or norbornyl;
a heterocyclic group such as any of those heteroaryl groups referred to above as examples of ring A, or a non aromatic heterocyclic group such as one having formula E defined above; or
a radical —CH₂[O(CH₂)_{w}]ₓZ² wherein Z² is H, —OH or —OCH₃ wherein x and w are independently 1, 2 or 3. Such radicals include the polyether radicals —O—(CH₂)₁₋₃OH, —O—(CH₂)₁₋₃O(C₁-C₃alkyl), —O—(CH₂)₁₋₃—O—(CH₂)₁₋₃OH, and —O—(CH₂)₁₋₃—O—(CH₂)₁₋₃O(C₁-C₃alkyl).

Case (c) arises when p is 1, and q is 1, and r is 0 or 1 and Z¹ is —NR^{A} or —C(═O)N(R^{A})—* wherein the bond marked * is attached to Q¹. In this case (c):
R^{A} when other than hydrogen may be, for example, methyl, ethyl, n- or iso-propyl, or trifluoromethyl; and
Q¹ is a radical —CH₂[O(CH₂)_{w}]ₓZ² wherein Z² is H, —OH or —O(C₁-C₃alkyl) wherein x and w are independently 1, 2 or 3. Such radicals include the polyether radicals —O—(CH₂)₁₋₃OH, —O—(CH₂)₁₋₃O(C₁-C₃alkyl), —O—(CH₂)₁₋₃—O—(CH₂)₁₋₃OH, and —O—(CH₂)₁₋₃—O—(CH₂)₁₋₃O(C₁-C₃alkyl).

Case (d) arises when p is 0, and q is 1, and r is 0 or 1 and Z¹ is —O— or —NR^{A}—. In this case (d) Q¹ is (i) hydrogen or an optional substituent which is not linked to -(Alk³)ₚ-(Z¹)_{q}-(Alk⁴)ᵣ- through a nitrogen atom; or (ii) Q¹ and R^{A}, taken together with the nitrogen to which they are attached form an optionally substituted heterocyclic ring of 5 or 6 ring atoms; or (iii) a radical —CH₂[O(CH₂)_{w}]ₓZ² wherein Z² is H, —OH or —OCH₃ wherein x and w are independently 1, 2 or 3. In this case (d) R^{A} when other than hydrogen may be, for example, methyl, ethyl, n- or iso-propyl, or trifluoromethyl; and Q¹ may be, for example:
a non-amino optional substituent, for example chloro, C₁-C₃alkoxy, cyano or acetyl; or a cyclopropyl, cyclopentyl or cyclohexyl group;
a radical —CH₂[O(CH₂)_{w}]ₓZ² wherein Z² is H, —OH or —OCH₃ wherein x and w are independently 1, 2 or 3. Such radicals include the polyether radicals —O—(CH₂)₁₋₃OH, —O—(CH₂)₁₋₃O(C₁-C₃alkyl), —O—(CH₂)₁₋₃—O—(CH₂)₁₋₃OH, and —O—(CH₂)₁₋₃—O—(CH₂)₁₋₃O(C₁-C₃alkyl), or
Q¹ and R^{A}, taken together with the nitrogen to which they are attached form an optionally substituted heterocyclic ring of 5 or 6 ring atoms, for example a cyclic amino group such as morpholino, piperidinyl, piperazinyl or methylpiperidinyl or a fluoro substituted cyclic amino group such as those of formulae (A)-(D):

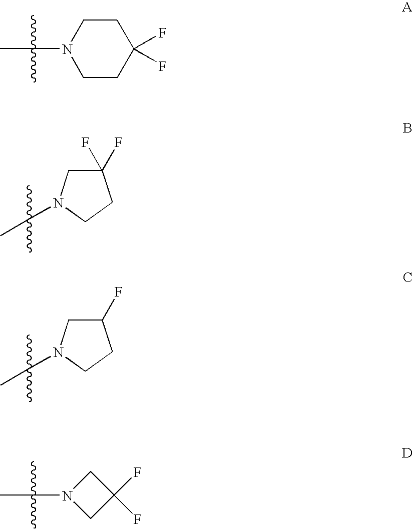

Case (e) arises when p is 0 or 1, q is 0, and r is 0 or 1. In this case, Q¹ is (i) hydrogen or an optional substituent which is not linked to -(Alk³)ₚ-(Z¹)_{q}-(Alk⁴)ᵣ- through a nitrogen atom or (ii) an optionally substituted carbocyclic radical; or (iii) an optionally substituted heterocyclic of 5 or 6 ring atoms which is not linked to -(Alk³)ₚ-(Z¹)_{q}-(Alk⁴)ᵣ- through a ring nitrogen; or (iv) a radical —CH₂[O(CH₂)_{w}]ₓZ² wherein Z² is H, —OH or —OCH₃ wherein x and w are independently 1, 2 or 3. In this case (e), Q¹ may be, for example:
a non-amino optional substituent, for example chloro, C₁-C₃alkoxy, cyano or acetyl; or a cyclopropyl, cyclopentyl or cyclohexyl group;
a heterocyclic group such as any of those heteroaryl groups referred to above as examples of ring A, or a non aromatic heterocyclic group such as one having formula E defined above; or
a radical —CH₂[O(CH₂)_{w}]ₓZ² wherein Z² is H, —OH or —OCH₃ wherein x and w are independently 1, 2 or 3. Such radicals include the polyether radicals —O—(CH₂)₁₋₃OH, —O—(CH₂)₁₋₃O(C₁-C₃alkyl), —O—(CH₂)₁₋₃—O—(CH₂)₁₋₃OH, and —O—(CH₂)₁₋₃—O—(CH₂)₁₋₃O(C₁-C₃alkyl).

The Group S₁ in Compounds (IC)

In compounds (IC) of the invention, it is presently preferred that S₁ be in the meta position of the ring.

As in the case of compounds (I), in a first subset of compounds (IC) of the invention, S₁ is hydrogen, or a substituent selected from fluoro, chloro, bromo, cyano (—CN), C₁-C₃alkyl optionally substituted with one or more fluorine substituents, C₁-C₃alkoxy optionally substituted with one or more fluorine substituents, —CH═CH₂, —C≡CH, cyclopropyl and —NH₂, or S₁ and R₃, or S₁ and R₄, together represent methylenedioxy (—OCH₂O—) or ethylenedioxy (—OCH₂CH₂O—) in either of which one or more hydrogens are optionally replaced by fluorine;

In a second subset of compounds (IC) of the invention, S₁ is a radical of formula (IB):

-(Alk³)ₚ-(Z¹)_{q}-(Alk⁴)ᵣ-Q¹ (IB)

wherein
p, q and r are independently 0 or 1;
Z¹ is —O—, —S—, —(C═O)—, —(C═S)—, —S(O)—, —SO₂—, —NR^{A}—, or, in either orientation, —C(═O)N(R^{A})— or —SO₂NR^{A}—;
Q¹ is (i) hydrogen or an optional substituent; or (ii) an optionally substituted carbocyclic or heterocyclic radical; or (iii) a radical —CH₂[O(CH₂)_{w}]ₓZ² wherein Z² is H, —OH or —O(C₁-C₃alkyl) wherein x and w are independently 1, 2 or 3;
R^{A} is hydrogen or C₁-C₃ alkyl optionally substituted with one or more fluorine substituents; and
Alk³ and Alk⁴ are divalent C₁-C₃ alkylene or C₂-C₃ alkenylene radicals, each optionally substituted by one or two substituents selected from fluoro, chloro, C₁-C₃alkyl optionally substituted with one or more fluorine substituents, C₁-C₃alkoxy optionally substituted with one or more fluorine substituents.

In compounds (IC) of the invention, when S₁ is a radical of formula (IB), Z¹, Q¹, R^{A}, Alk³ and Alk₄ therein may be any of those radicals or groups defined and discussed in relation to compounds (I), cases (a), (b), (c), (d) or (e) above.

One particular sub-class of compounds of the invention consists of those of formula (ID):

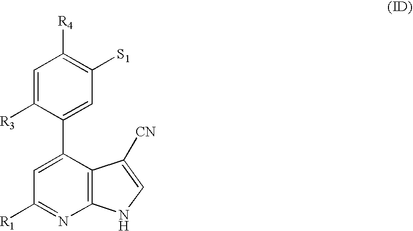

and salts, N-oxides, hydrates, or solvates thereof, wherein:
R₁ and S₁ are independently selected from (a) hydrogen, methoxy, ethoxy, methylthio or ethylthio; (b) a group of formula —X¹-Alk⁵-(CO)_{w}NR^{C}R^{D} wherein w is 0 or 1, X¹ is —O— or —S—, Alk⁵ is a straight or branched chain C₁-C₃alkylene radical, R^{C} is C₁-C₃alkyl and R^{D} is C₁-C₃alkyl or hydroxyl (C₁-C₃alkyl)-; and (c) a group of formula —X¹-Alk⁵-Ar wherein X¹ is —O— or —S—, Alk⁵ is a straight or branched chain C₁-C₃alkylene radical, and Ar is phenyl or a 5- or 6-membered heteroaryl ring wherein at least one hetero atom is nitrogen; PROVIDED THAT R₁ and S₁ are not both hydrogen;
R₃ is fluoro, chloro, bromo or methyl; and
R₄ is fluoro, chloro, bromo methyl, ethyl, isopropyl, methoxy, or cyano.

Specific examples of compounds of the invention include those of the Examples herein.

There are multiple synthetic strategies for the synthesis of the compounds (I) with which the present invention is concerned, but all rely on known chemistry, known to the synthetic organic chemist. Thus, compounds according to formula (I) or (IC) can be synthesised according to procedures described in the standard literature and are well-known to the one skilled in the art. Typical literature sources are “Advanced organic chemistry”, 4th Edition (Wiley), J March, “Comprehensive Organic Transformation”, 2^{nd} Edition (Wiley), R. C. Larock, “Handbook of Heterocyclic Chemistry”, 2^{nd} Edition (Pergamon), A. R. Katritzky), review articles such as found in “Synthesis”, “Acc. Chem. Res.”, “Chem. Rev”, or primary literature sources identified by standard literature searches online or from secondary sources such as “Chemical Abstracts” or “Beilstein”. Such literature methods include those of the preparative Examples herein, and methods analogous thereto. In general, compounds of the invention may be prepared by methods as described in the Examples herein, or by methods analogous to those described ion the Examples herein.

The compounds of the invention are inhibitors of HSP90 and are useful in the treatment of diseases which are responsive to inhibition of HSP90 activity such as cancers; viral diseases such as Hepatitis C (HCV) (Waxman, 2002); Immunosuppression such as in transplantation (Bijimakers, 2000 and Yorgin, 2000); Anti-inflammatory diseases (Bucci, 2000) such as Rheumatoid arthritis, Asthma, MS, Type I Diabetes, Lupus, Psoriasis and Inflammatory Bowel Disease; Cystic fibrosis (Fuller, 2000); Angiogenesis-related diseases (Hur, 2002 and Kurebayashi, 2001): diabetic retinopathy, haemangiomas, psoriasis, endometriosis and tumour angiogenesis. Also an Hsp90 inhibitor of the invention may protect normal cells against chemotherapy-induced toxicity and be useful in diseases where failure to undergo apoptosis is an underlying factor. Such an Hsp90 inhibitor may also be useful in diseases where the induction of a cell stress or heat shock protein response could be beneficial, for example, protection from hypoxia-ischemic injury due to elevation of Hsp70 in the heart (Hutter, 1996 and Trost, 1998) and brain (Plumier, 1997 and Rajder, 2000). An Hsp90 inhibitor-induced increase in Hsp70 levels could also be useful in diseases where protein misfolding or aggregation is a major causal factor, for example, neurogenerative disorders such as scrapie/CJD, Huntingdon's and Alzheimer's (Sittler, 2001; Trazelt, 1995 and Winklhofer, 2001)”.

Accordingly, the invention also includes:

(i) A pharmaceutical or veterinary composition comprising a compound of formula (I) or (IC) above, together with a pharmaceutically or veterinarily acceptable carrier.

(ii) The use of a compound a compound of formula (I) or (IC) above in the preparation of a composition for composition for inhibition of HSP90 activity in vitro or in vivo.

(iii). A method of treatment of diseases or conditions which are responsive to inhibition of HSP90 activity in mammals which method comprises administering to the mammal an amount of a compound of formula (I) or (IC) above effective to inhibit said HSP90 activity.

It will be understood that the specific dose level for any particular patient will depend upon a variety of factors including the activity of the specific compound employed, the age, body weight, general health, sex, diet, time of administration, route of administration, rate of excretion, drug combination and the causative mechanism and severity of the particular disease undergoing therapy. In general, a suitable dose for orally administrable formulations will usually be in the range of 0.1 to 3000 mg, once, twice or three times per day, or the equivalent daily amount administered by infusion or other routes. However, optimum dose levels and frequency of dosing will be determined by clinical trials as is conventional in the art.

The compounds with which the invention is concerned may be prepared for administration by any route consistent with their pharmacokinetic properties. The orally administrable compositions may be in the form of tablets, capsules, powders, granules, lozenges, liquid or gel preparations, such as oral, topical, or sterile parenteral solutions or suspensions. Tablets and capsules for oral administration may be in unit dose presentation form, and may contain conventional excipients such as binding agents, for example syrup, acacia, gelatin, sorbitol, tragacanth, or polyvinyl-pyrrolidone; fillers for example lactose, sugar, maize-starch, calcium phosphate, sorbitol or glycine; tabletting lubricant, for example magnesium stearate, talc, polyethylene glycol or silica; disintegrants for example potato starch, or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives such as suspending agents, for example sorbitol, syrup, methyl cellulose, glucose syrup, gelatin hydrogenated edible fats; emulsifying agents, for example lecithin, sorbitan monooleate, or acacia; non-aqueous vehicles (which may include edible oils), for example almond oil, fractionated coconut oil, oily esters such as glycerine, propylene glycol, or ethyl alcohol; preservatives, for example methyl or propyl p-hydroxybenzoate or sorbic acid, and if desired conventional flavouring or colouring agents.

For topical application to the skin, the drug may be made up into a cream, lotion or ointment. Cream or ointment formulations which may be used for the drug are conventional formulations well known in the art, for example as described in standard textbooks of pharmaceutics such as the British Pharmacopoeia.

The active ingredient may also be administered parenterally in a sterile medium. Depending on the vehicle and concentration used, the drug can either be suspended or dissolved in the vehicle. Advantageously, adjuvants such as a local anaesthetic, preservative and buffering agents can be dissolved in the vehicle.

Compounds of the invention may be administered together with other classes of pharmaceutically active drugs. For example, for the treatment of cancers, combination therapy with two or more different classes of anticancer agent is a recognised and widespread practice. The present compounds may be used in such combination therapy, particularly where the other drug(s) have a mode of action different from HSP90 inhibition.

The following examples illustrate the preparation and activities of specific compounds of the invention and are not intended to be limiting of the full scope of the invention.

General Procedures

All reagents obtained from commercial sources were used without further purification. Anhydrous solvents were obtained from commercial sources and used without further drying. Flash chromatography was performed with pre-packed silica gel cartridges (Strata SI-1; 61 Å, Phenomenex, Cheshire UK or IST Flash II, 54 Å, Argonaut, Hengoed, UK). Thin layer chromatography was conducted with 5×10 cm plates coated with Merck Type 60 F₂₅₄ silica gel.

The compounds of the present invention were characterized by Liquid chromatography/mass spectrometry (LCMS) using a Hewlett Packard 1100 series LCMS/MSD linked to quadripole detector (ionization mode: electron spray positive or negative; column: Phenomenex Luna 3 u C18(2) 30×4.6 mm; Buffer A prepared by dissolving 1.93 g ammonium acetate in 2.5 L HPLC grade H₂O and adding 2 mL formic acid. Buffer B prepared by adding 132 mL buffer A to 2.5 L of HPLC grade acetonitrile and adding 2 mL formic acid; elution gradient 95:5 to 5:95 buffer A: buffer B over 3.75 minutes or 7.5 minutes. Flow rate=2.0 mL/min. Retention Times are reported in minutes. Ionisation is positive unless otherwise stated.

Nuclear magnetic resonance (NMR) analysis was performed with a Brucker DPX-400 MHz NMR spectrometer. The spectral reference was the known chemical shift of the solvent. Proton NMR data is reported as follows: chemical shift (δ) in ppm, multiplicity (s=singlet, d=doublet, t=triplet, q=quartet, p=pentet, m=multiplet, dd=doublet of doublet, br=broad), integration, coupling constant.

Some compounds of the invention were purified by preparative HPLC. Preparative HPLC purifications were performed on a Waters FractionLynx MS Autopurification system with a Gemini® 5 μM C18(2), 100 mm×20 mm i.d. column from Phenomenex, running at a flow rate of 20 mL min⁻¹ with UV diode array detection (210-400 nm) and mass-directed collection. Gradients used for each compound are shown in Table 1.

At pH 4: Solvent A: L HPLC grade Water+10 mM ammonium acetate+0.08% v/v formic acid.

Solvent B: 95% v/v HPLC grade acetonitrile+5% v/v Solvent A+0.08% v/v formic acid.

At pH 9: Solvent A: HPLCS grade Water+10 mM ammonium acetate+0.08% v/v ammonia solution.

Solvent B: 95% v/v HPLC grade acetonitrile+5% v/v Solvent A+0.08% v/v ammonia solution.

The mass spectrometer was a Waters Micromass ZQ2000 spectrometer operating in positive or negative ion electrospray ionisation modes, with a molecular weight scan range of 150 to 1000.

TABLE 1
Preparative HPLC gradients

% B for Compound no.

| Time/min | 8 | 9 | 11 | 12 |
|---|---|---|---|---|
| 0.0 | 5 | 5 | 5 | 5 |
| 0.5 | 20 | 25 | 30 | 35 |
| 7.0 | 40 | 45 | 50 | 55 |
| 7.5 | 95 | 95 | 95 | 95 |
| 9.5 | 95 | 95 | 95 | 95 |
| 10 | 5 | 5 | 5 | 5 |

IUPAC chemical names were generated using AutoNom Standard.

Some compounds of the invention can be made (by way of example) by following the route outlined in scheme 1. Experimental Methods, reagents and product isolation methods will be known to those skilled in the art of organic synthesis. It is understood that other methods can also be used.

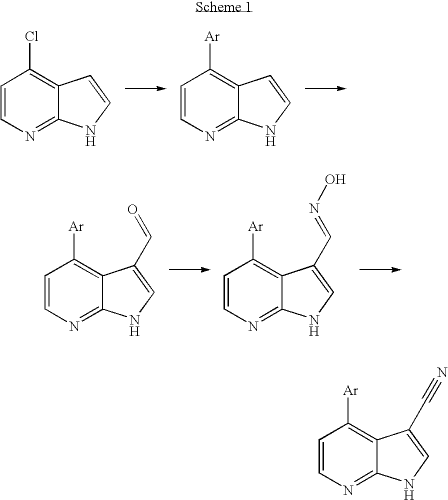

### Example 1

### 4-(2,4-Dimethyl-phenyl)-1H-pyrrolo[2,3-b]pyridine-3-carbonitrile

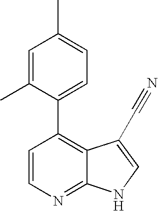

Step 1.

### 4-(2,4-Dimethyl-phenyl)-1H-pyrrolo[2,3-b]pyridine

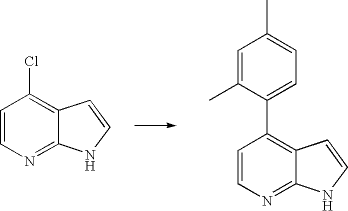

Bis(tri-t-butylphosphine)palladium(0) was added to a suspension of 4-Chloro-1H-Pyrrolo[2,3-b]pyridine, 2,4-Dimethylphenylboronic acid and potassium fluoride in 1,4-Dioxan under a nitrogen atmosphere. The reaction mixture heated at ˜80° C., for ˜2 hrs. The suspension was allowed to cool and diluted with ethyl acetate, the phases were separated and the organic phase was washed with water, saturated aqueous sodium chloride solution, then dried over anhydrous sodium sulphate and concentrated to a pale yellow solid. The crude product was purified by column chromatography on silica gel, eluting with mixtures of dichloromethane and methanol. The resulting product was re-purified by column chromatography on silica gel, eluting with mixtures of ethyl acetate and hexane, to afford title compound as a pale yellow solid.

LCMS retention time=2.468 minutes; m/z=233.1 [M+H]⁺ (Run time 3.75 mins)

Step 2

### 4-(2,4-Dimethyl-phenyl)-1H-pyrrolo[2,3-b]pyridine-3-carbaldehyde

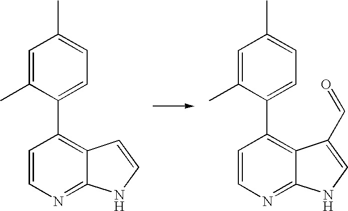

DMF was added slowly to cooled (ice-water bath) phosphorous oxychloride and the mixture stirred. 4-(2,4-Dimethyl-phenyl)-1H-pyrrolo[2,3-b]pyridine was added and the suspension heated at 110° C., for 18 hrs. The resulting suspension was allowed to cool and water added the mixture was then neutralised with aqueous sodium hydroxide and extracted with ethyl acetate. The organic extracts were washed with water and saturated aqueous sodium chloride solution, the solution was dried over anhydrous sodium sulphate and concentrated to a pale yellow solid. The crude product was purified by column chromatography on silica gel, eluting with mixtures of dichloromethane and methanol. The crude product was re-purified by column chromatography on silica gel, eluting with mixtures of ethyl acetate and hexane, to give a pale yellow solid.

LCMS retention time=2.22 minutes; m/z=251.1 [M+H]⁺ (Run time 3.75 mins)

Step 3

### 4-(2,4-Dimethyl-phenyl)-1H-pyrrolo[2,3-b]pyridine-3-carbaldehyde oxime

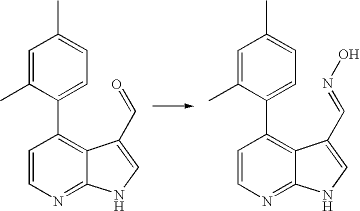

Hydroxylamine hydrochloride was added to a suspension of 4-(2,4-Dimethyl-phenyl)-1H-pyrrolo[2,3-b]pyridine-3-carbaldehyde in ethanol. Sodium acetate was added and the suspension heated at ˜90° C., for ˜90 mins. The suspension was allowed to cool and concentrated, water was added and the mixture extracted with ethyl acetate. The organic extracts were washed with water and saturated aqueous sodium chloride solution, the solution was dried over anhydrous sodium sulphate and concentrated to a pale brown solid.

LCMS retention time=2.147 minutes; m/z=266.1 [M+H]⁺ (Run time 3.75 mins)

Step 5

### 4-(2,4-Dimethyl-phenyl)-1H-pyrrolo[2,3-b]pyridine-3-carbonitrile

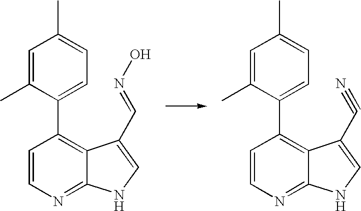

Thionyl chloride was added to a suspension of 4-(2,4-Dimethyl-phenyl)-1H-pyrrolo[2,3-b]pyridine-3-carbaldehyde oxime in dichloromethane, at ˜0° C. (ice/water), the resulting solution was stirred at room temperature for ˜60 mins. to give an off-white suspension. The suspension was concentrated in vacuo and saturated aqueous sodium hydrogen carbonate solution added the mixture extracted with ethyl acetate, the extracts were washed with water and saturated aqueous sodium chloride solution, the solution was dried over anhydrous sodium sulphate and concentrated to a pale brown solid. The crude product was re-purified by preparative HPLCMS, to give the product as an off-white solid.

LCMS retention time=2.262 minutes; m/z=248.1 [M+H]⁺ (Run time 3.75 mins)

¹H NMR (400 MHz, DMSO-d₆) δ 2.06 (s, 3H), 2.35 (s, 3H), 7.07 (d, 1H, J=5.0 Hz), 7.11 (m, 2H), 7.12 (brs, 1H), 8.41 (d, 1H, J=5.0 Hz), 8.42 (s, 1H), 12.89 (brs, 1H).

This compound had activity ‘A’ in the fluorescence polarization assay described below.

Some compounds of the invention can be made (by way of example) by following the route outlined in scheme 2. Experimental Methods, reagents and product isolation methods will be known to those skilled in the art of organic synthesis. It is understood that other methods can also be used.

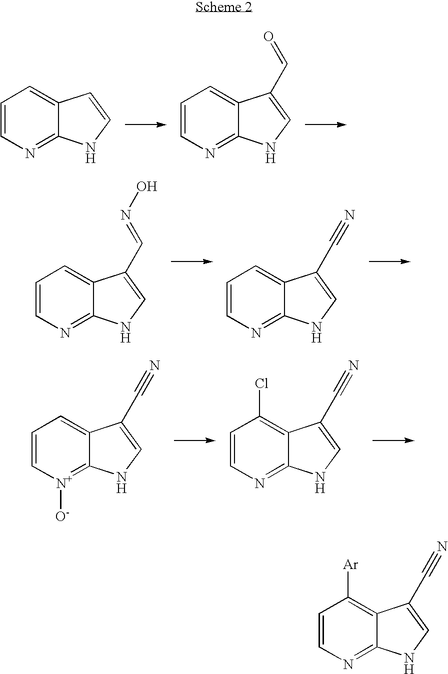

### Example 2

### 4-(2,4-Dichloro-phenyl)-1H-pyrrolo[2,3-b]pyridine-3-carbonitrile

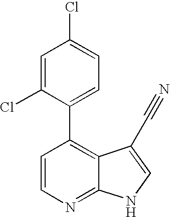

Step 1

### 1H-Pyrrolo[2,3-b]pyridine-3-carbaldehyde

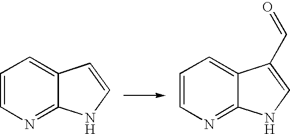

7-Azaindole was added to a solution of hexamethylenetetramine (2 equivalents) in water/acetic acid mixture (1:2). The resulting solution was heated under reflux for ˜3 hrs. to give a dark yellow solution. The solution was allowed to cool and poured into water, to give a white suspension. Solids were removed by filtration and washed with water, to give an off-white powder after being dried in vacuo.

LCMS retention time=1.366 minutes; m/z=147.0 [M+H]⁺ (Run time 3.75 mins)

Step 2

### 1H-Pyrrolo[2,3-b]pyridine-3-carbaldehyde oxime

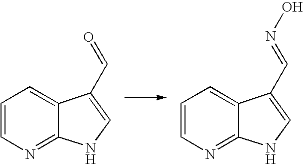

Aqueous hydroxylamine was added to a suspension of the 1H-pyrrolo[2,3-b]pyridine-3-carbaldehyde in water and the suspension heated to 100° C. for ˜60 mins to give an off-white suspension. The suspension was allowed to cool to ambient temperature and then further cooled (ice/water bath). Solids were removed by filtration and washed with water and dried in vacuo.

LCMS retention time=1.273 minutes; m/z=162.0 [M+H]⁺ (Run time 3.75 mins)

Step 3

### 1H-Pyrrolo[2,3-b]pyridine-3-carbonitrile

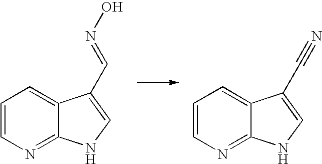

A suspension of the 1H-pyrrolo[2,3-b]pyridine-3-carbaldehyde oxime in acetic anhydride was heated to 140° C., for ˜3 hrs. The resulting solution was allowed to cool and poured into water to give an off-white suspension, the solids were removed by filtration and washed with water, the crude solid was re-crystallised from boiling water to give an off-white solid. Solids were removed by filtration and washed with water to give a pale brown powder and dried in vacuo.

LCMS retention time=1.578 minutes, m/z=144.1 [M+H]⁺ (Run time 3.75 mins)

Step 4

### 7-Oxy-1H-pyrrolo[2,3-b]pyridine-3-carbonitrile

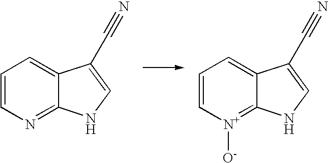

A suspension of the 1H-pyrrolo[2,3-b]pyridine-3-carbonitrile in chloroform was cooled (ice/water bath). m-Chloroperoxybenzoic acid was added and the suspension stirred, room temperature for ˜18 hrs. to give an off-white suspension. Methanesulphonic acid (1.5 equivalents) was added to give a pale yellow solution, diethyl ether was then added and the solution cooled (ice/water) to afford an off-white precipitate. Solids were removed by filtration and washed with diethyl ether and dried in vacuo to afford the product as the methanesulphonate salt.

LCMS retention time=1.085 minutes; m/z=160.1 [M+H]⁺ (Run time 3.75 mins)

Step 5

### 4-Chloro-1H-pyrrolo[2,3-b]pyridine-3-carbonitrile

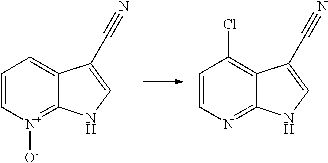

Methanesulphonyl chloride (2 equivalents) was added to a suspension of the 7-Oxy-1H-pyrrolo[2,3-b]pyridine-3-carbonitrile methanesulphonate salt in DMF and the suspension heated, 80° C., for ˜2 hrs. to give a pale brown solution. The resulting solution was allowed to cool and poured into water to give a pale yellow suspension. The solids were removed by filtration and washed with water and hexane, to give an off-white solid which was dried in vacuo.

LCMS retention time=1.841 minutes; m/z=178.0 [M+H]⁺ (Run time 3.75 mins)

Step 6

### 4-(2,4-Dichloro-phenyl)-1H-pyrrolo[2,3-b]pyridine-3-carbonitrile

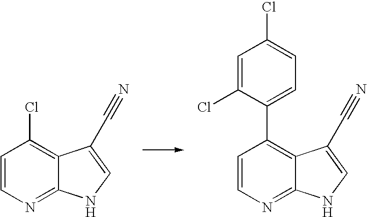

Bis(tri-t-butylphosphine)palladium(0) was added to a suspension of 4-Chloro-1H-pyrrolo[2,3-b]pyridine-3-carbonitrile, 2,4-Dichlorophenylboronic acid and potassium fluoride in 1,4-Dioxan under a nitrogen atmosphere. The mixture heated at ˜95° C., for ˜24 hrs. The suspension was allowed to cool and diluted with ethyl acetate, the mixture washed with water and saturated aqueous sodium chloride solution, the solution was dried over anhydrous sodium sulphate and concentrated to a pale yellow solid. The crude product was purified by column chromatography on silica gel, eluting with mixtures of dichloromethane and methanol. The crude product was re-purified by preparative HPLC (pH 4), to give the product as a pale brown solid.

LCMS retention time=2.315 minutes; m/z=288.0/290.0 [M+H]⁺ (Run time 3.75 mins)

¹H NMR (400 MHz, DMSO-d₆) δ 7.20 (d, 1H, J=4.8 Hz), 7.51 (d, 1H, J=8.2 Hz), 7.58 (dd, 1H, J=8.2, 2.0 Hz), 7.83 (d, 1H, J=2.0 Hz), 8.48 (d, 1H, J=5.0 Hz), 13.03 (brs, 1H)

This compound had activity ‘A’ in the fluorescence polarization assay described below.

### Example 3

### 6-Chloro-4-(2,4-dichloro-phenyl)-1H-pyrrolo[2,3-b]pyridine-3-carbonitrile

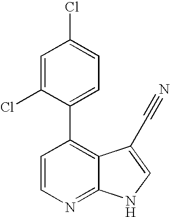

Step 1

### 4-(2,4-Dichloro-phenyl)-7-Oxy-1H-pyrrolo[2,3-b]pyridine-3-carbonitrile

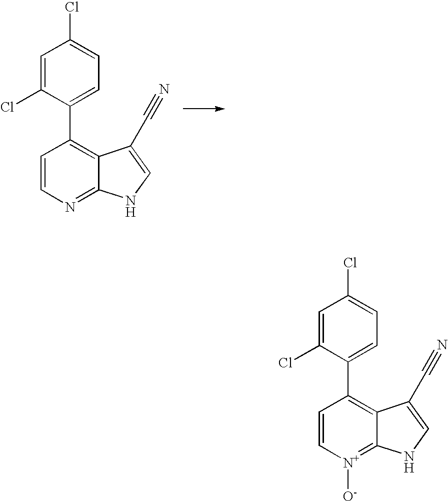

A suspension of the 4-(2,4-dichloro-phenyl)-1H-pyrrolo[2,3-b]pyridine-3-carbonitrile in chloroform was, cooled (ice/water). m-Chloroperoxybenzoic acid was added and the suspension stirred at room temperature for ˜18 hrs. The resulting solution was concentrated to an orange gum. The crude product was purified by column chromatography, silica, eluting with mixtures of dichloromethane and methanol.

LCMS retention time=1.973 minutes; m/z=304.0/306.0 [M+H]⁺ (Run time 3.75 mins)

Step 2

### 6-Chloro-4-(2,4-dichloro-phenyl)-1H-pyrrolo[2,3-b]pyridine-3-carbonitrile

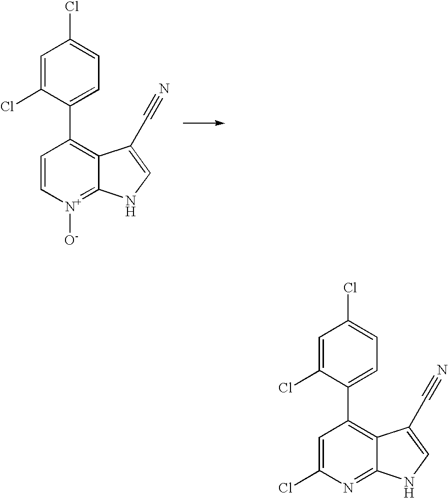

Methanesulphonyl chloride (2 equivalents) was added to a suspension of the 4-(2,4-Dichloro-phenyl)-7-Oxy-1H-pyrrolo[2,3-b]pyridine-3-carbonitrile in DMF and the suspension heated at 80° C., for ˜2 hrs. to give a pale brown solution. The resulting solution was allowed to cool and poured into water, the mixture was extracted with ethyl acetate and the extracts were washed with water and saturated aqueous sodium chloride solution, the solution was dried over anhydrous sodium sulphate and concentrated to a pale brown solid. The crude product was purified by preparative HPLC (pH4) to give the product as an off-white solid.

LCMS retention time=2.577 minutes; m/z=322 [M+H]⁺ (Run time 3.75 mins)

¹H NMR (400 MHz, DMSO-d₆) δ 7.36 (s, 1H), 7.55 (d, 1H, J=8.1 Hz), 7.60 (dd, 1H, J=8.1, 2.0 Hz), 7.85 (d, 1H, J=2.0 Hz), 8.53 (brs, 1H), 13.34 (brs, 1H)

This compound had activity ‘A’ in the fluorescence polarization assay described below.

### Example 4

### 4-(2,4-Dichloro-5-methoxy-phenyl)-1H-pyrrolo[2,3-b]pyridine-3-carbonitrile

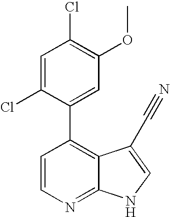

This compound was made by way of the route outlined in scheme 2, with the exception of the pyrrolo nitrogen being protected prior to the cross coupling step.

Step 1

### 4-Chloro-1-(2-trimethylsilanyl-ethoxymethyl)-1H-pyrrolo[2,3-b]pyridine-3-carbonitrile

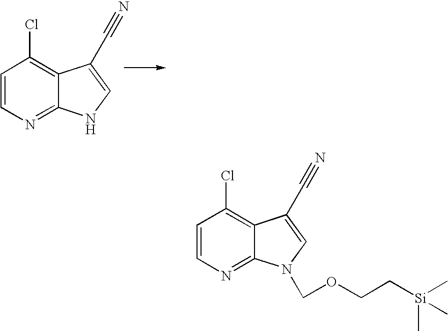

Sodium hydride (60% dispersion in mineral oil) was added to a cooled (ice/water bath) suspension of 4-Chloro-1H-pyrrolo[2,3-b]pyridine-3-carbonitrile in THF and the mixture stirred for ˜60 mins. 2-(Trimethylsilyl)ethoxymethyl chloride was added and the mixture stirred for ˜2 hrs. The suspension was diluted with ethyl acetate and the mixture washed with water and saturated aqueous sodium chloride solution, the solution was dried over anhydrous sodium sulphate and concentrated to a pale brown gum. The crude product was purified by column chromatography on silica gel, eluting with mixtures of ethyl acetate and hexane.

LCMS retention time=2.728 minutes; m/z=308.1 [M+H]⁺ (Run time 3.75 mins)

Step 2

### 4-(2,4-Dichloro-5-methoxy-phenyl)-1-(2-trimethylsilanyl-ethoxymethyl)-1H-pyrrolo[2,3-b]pyridine-3-carbonitrile

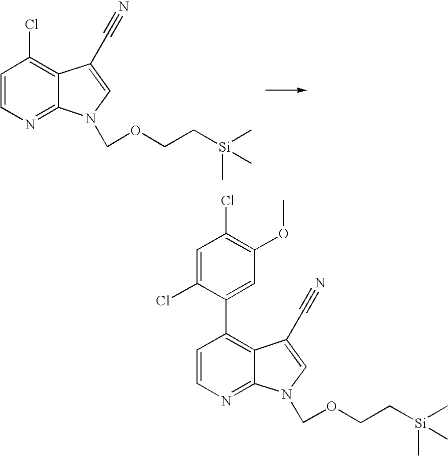

1,1′-Bis(diphenylphosphino)ferrorecene palladium(II) chloride was added to a suspension of 4-chloro-1-(2-trimethylsilanyl-ethoxymethyl)-1H-pyrrolo[2,3-b]pyridine-3-carbonitrile and 2,4-Dichloro-5-methoxyphenylboronic acid in 1,4-Dioxan under a nitrogen atmosphere. Aqueous potassium phosphate (2M) was added and the mixture heated at ˜95° C., for ˜24 hrs. The suspension was allowed to cool and diluted with ethyl acetate, the mixture washed with water and saturated aqueous sodium chloride solution, the solution was dried over anhydrous sodium sulphate and concentrated to a dark brown gum. The crude product was purified by column chromatography, silica, eluting with mixtures of ethyl acetate and hexane.

LCMS retention time=2.863 minutes; m/z=448.1/450.1 [M+H]⁺ (Run time 3.75 mins)

Step 3

### 4-(2,4-Dichloro-5-methoxy-phenyl)-1H-pyrrolo[2,3-b]pyridine-3-carbonitrile

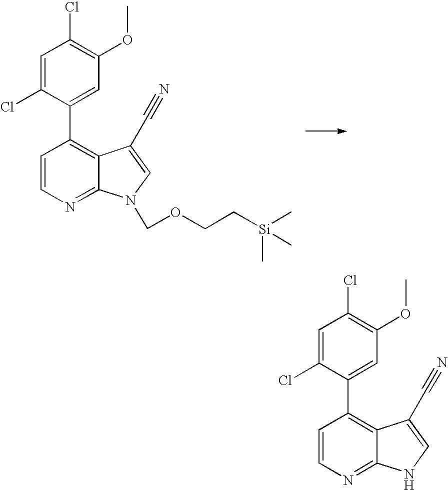

Tetrabutylammonium fluoride (1M in THF) was added to a solution of 4-(2,4-dichloro-5-methoxy-phenyl)-1-(2-trimethylsilanyl-ethoxymethyl)-1H-pyrrolo[2,3-b]pyridine-3-carbonitrile and ethylenediamine (1.5 equivalents) in THF, under a nitrogen atmosphere and the solution heated at ˜65° C., for ˜18 hrs. The solution was allowed to cool and diluted with ethyl acetate. The phases were separated and the organic phase was washed with water, saturated aqueous sodium chloride solution then dried over anhydrous sodium sulphate and concentrated to a pale yellow solid. The crude product was purified by column chromatography on silica gel, eluting with mixtures of ethyl acetate and hexane.

LCMS retention time=2.310 minutes; m/z=318.1/320.05 [M+H]⁺ (Run time 3.75 mins)

¹H NMR (400 MHz, DMSO-d₆) δ 3.59 (s, 3H), 7.25 (d, 1H, J=4.8 Hz), 7.29 (s, 1H), 7.78 (s, 1H), 8.48 (d, 1H, J=4.8 Hz), 8.50 (s, 1H), 13.03 (brs, 1H).

This compound had activity ‘A’ in the fluorescence polarization assay described below.

### Example 5

### 6-Chloro-4-(2,4-dichloro-5-methoxy-phenyl)-1H-pyrrolo[2,3-b]pyridine-3-carbonitrile

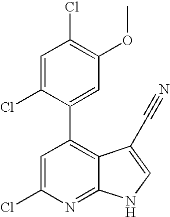

Step 1

### 4-(2,4-Dichloro-5-methoxy-phenyl)-7-Oxy-1H-pyrrolo[2,3-b]pyridine-3-carbonitrile

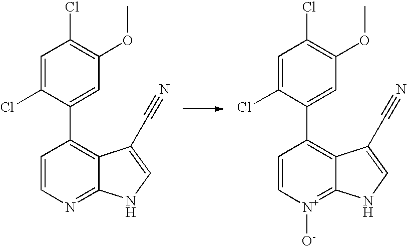

A suspension of the 4-(2,4-dichloro-5-methoxy-phenyl)-1H-pyrrolo[2,3-b]pyridine-3-carbonitrile in chloroform was cooled (ice/water). m-Chloroperbenzoic acid was added and the suspension stirred at room temperature for ˜18 hrs. The resulting solution was concentrated to an orange gum and this crude product was purified by column chromatography on silica gel, eluting with mixtures of dichloromethane and methanol.

LCMS retention time=1.967 minutes; m/z=334.0/336.0 [M+H]⁺ (Run time 3.75 mins)

Step 2

### 6-Chloro-4-(2,4-dichloro-5-methoxy-phenyl)-1H-pyrrolo[2,3-b]pyridine-3-carbonitrile

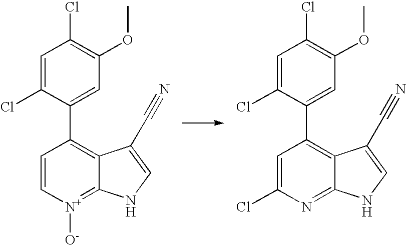

The title compound was prepared by way of the method outlined for example 3 step 2, product was purified by Preparative HPLC (pH4).

LCMS retention time=2.568 minutes; m/z=352 [M+H]⁺ (Run time 3.75 mins)

This compound had activity ‘A’ in the fluorescence polarization assay described below.

### Example 6

### 3-Bromo-4-(2,4-dimethyl-phenyl)-1H-pyrrolo[2,3-b]pyridine-3-carbonitrile

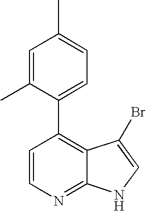

N-Bromosuccinimide was added to a solution of 4-(2,4-dimethyl-phenyl)-1H-pyrrolo[2,3-b]pyridine (example 1 step 1) in dichloromethane, and the solution stirred for ˜90 mins. The solution was diluted with dichloromethane and washed with water and saturated aqueous sodium chloride solution. The phases were separated dried over anhydrous sodium sulphate and concentrated to a red/orange gum. The crude product was purified by column chromatography on silica gel, eluting with mixtures of ethyl acetate and hexane. The crude product was re-purified by preparative HPLC (pH4), to give the product as a pale brown solid.

LCMS retention time=2.569 minutes; m/z=301.0/303.0 [M+H]⁺ (Run time 3.75 mins).

¹H NMR (400 MHz, DMSO-d₆) δ 1.96 (s, 3H), 2.34 (s, 3H), 6.86 (d, 1H, J=4.8 Hz), 7.06 (m, 2H), 7.11 (s, 1H), 7.64 (d, 1H, J=2.) Hz), 8.29 (d, 1H, J=4.8 Hz), 12.16 (brs, 1H).

This compound had activity ‘B’ in the fluorescence polarization assay described below.

### Example 7

### 4-(2,4-Dimethyl-phenyl)-1H-pyrrolo[2,3-b]pyridine-3-carbaldehyde

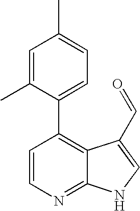

This compound was prepared by the methods outlined in example 1 steps 1 and 2, using 2,4-dimethyl-phenyl-boronic acid for step 1 (cross coupling).

LCMS retention time=2.22 minutes; m/z=251.1 [M+H]⁺ (Run time 3.75 mins).

This compound had activity ‘A’ in the fluorescence polarization assay described below.

### Example 8

### 4-(2,4-Difluorol-phenyl)-1H-pyrrolo[2,3-b]pyridine-3-carbaldehyde

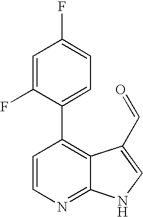

This compound was prepared by the methods outlined in example 1 steps 1 and 2, using 2,4-difluoro-phenyl-boronic acid for step 1 (cross coupling).

LCMS retention time=2.05 minutes; m/z=259 [M+H]⁺ (Run time 3.75 mins).

¹H NMR (400 MHz, DMSO-d₆) δ 7.18 (dd, 1H J=8.5, 2.6 Hz), 7.21 (d, 1H, J=4.8 Hz), 7.31 (dt, 1H, J=10, 2.6 Hz), 7.51 (m, 1H), 8.44 (d, 1H, J=4.8 Hz), 8.52 (s, 1H), 9.60 (s, 1H), 12.96 (brs, 1H).

### Example 9

### 6-(2-Diethylamino-ethoxy)-4-phenyl-1H-pyrrolo[2,3-b]pyridine-3-carbonitrile

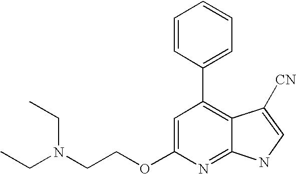

Step 1

### 5-Amino-1-benzhydryl-1H-pyrrole-3-carbonitrile

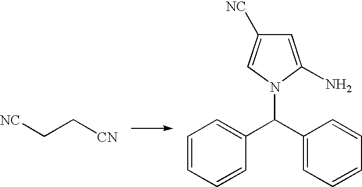

A solution of ethyl formate (4.0 mL) and succinonitrile (4.0 g, 49.9 mmol) in THF (80 mL) was added dropwise to an ice bath cooled solution of potassium tert butoxide in THF (50 mL) under a nitrogen atmosphere. The resulting suspension was stirred at ambient temperature for 30 minutes. The solvent was removed in vacuo and the residual solid was suspended in water (20 mL) and amino diphenyl methane was added dropwise. Acetic acid (20 mL) was then added and the reaction mixture was heated to 100° C. for 10 minutes. The mixture was cooled to ambient temperature and water (150 mL) was added and the mixture was extracted with diethyl ether. (200 mL) and the phases separated. The ether extracts were washed sequentially with 2.0 M aqueous sodium hydroxide solution (75 mL) water (2×75 mL) and saturated sodium chloride solution (75 mL). The organic phase was dried over sodium sulphate, filtered and the filtrate solvents evaporated in vacuo to leave a dark brown gum. The residue was dissolved in ethanol (50 mL) and sodium ethoxide (6.8 g) was added and the resulting solution stirred at ambient temperature for 60 minutes. Water (250 mL) was added and the mixture then extracted with diethyl ether (200 mL). The phases were separated and the organic phase washed with water (250 mL) and saturated sodium chloride solution (150 mL). The organic phase was dried over sodium sulphate, filtered and the filtrate solvents evaporated in vacuo to afford a dark-brown solid (8.1 g).

Step 2

### 1-Benzhydryl-6-hydroxy-4-phenyl-1H-pyrrolo[2,3-b]pyridine-3-carbonitrile

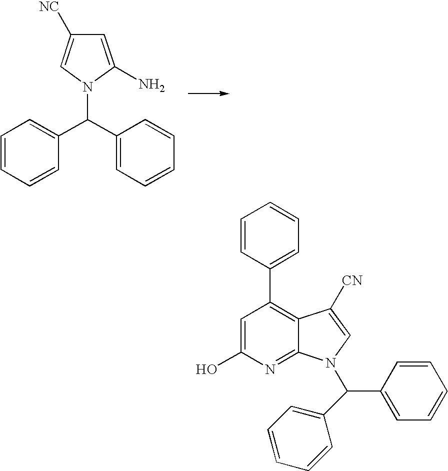

3-Oxo-3-phenyl-propionic acid ethyl ester (3.85 g) was added to a suspension of 5-amino-1-benzhydryl-1H-pyrrole-3-carbonitrile (5.5 g) in 1-butanol (80 mL) and the suspension stirred. Hydrochloric acid (6N, 1.0 mL) was added and the mixture was heated at 120° C., for 2 hrs and then the resulting solution was allowed to cool and concentrated to a dark purple gum. The crude product was purified by column chromatography on silica, gel eluting with dichloromethane to give the product as a pale-brown solid (3.25 g).

LCMS retention time=2.653 minutes; m/z=402.2 [M+H]⁺ (Run time 3.75 mins)

Step 3

### 1-Benzhydryl-6-(2-diethylamino-ethoxy)-4-phenyl-1H-pyrrolo[2,3-b]pyridine-3-carbonitrile

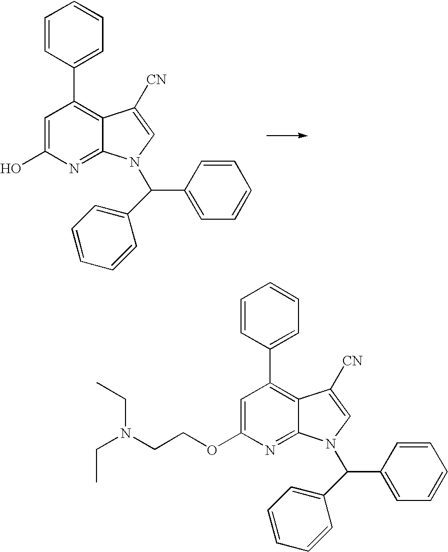

Cesium carbonate (3.25 g) was added to a solution of 1-Benzhydryl-6-hydroxy-4-phenyl-1H-pyrrolo[2,3-b]pyridine-3-carbonitrile (2.0 g) in acetone (30 mL) and the suspension stirred. 2-Bromo-N,N-diethylethylamine hydrochloride (2.0 g) was added and the suspension was heated at 70° C., for 90 mins. The resulting suspension was allowed to cool and ethyl acetate (250 mL) was added. The organic phase was washed with water (1×100 mL) then saturated aqueous sodium chloride solution (100 mL). The organic phase was dried over anhydrous sodium sulphate and concentrated to a dark brown gum (2.0 g) which solidified on standing.

LCMS retention time=2.106 minutes; m/z=501.3 [M+H]⁺ (Run time 3.75 mins)

Step 4

### 6-(2-Diethylamino-ethoxy)-4-phenyl-1H-pyrrolo[2,3-b]pyridine-3-carbonitrile

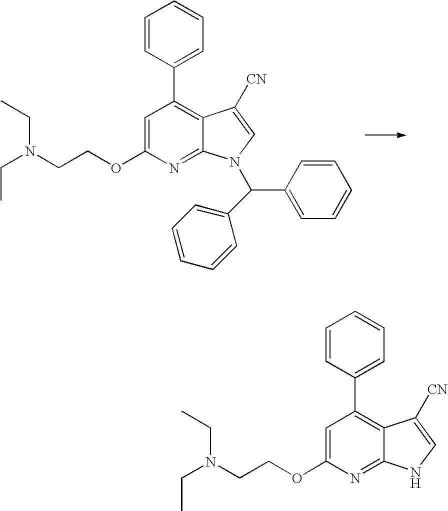

A solution of potassium tert-butoxide (1.0 g) in THF (20 mL) was added to a solution of 1-Benzhydryl-6-methoxy-4-phenyl-1H-pyrrolo[2,3-b]pyridine-3-carbonitrile in DMSO (2.5 mL) and the suspension stirred at ambient temperature for 60 mins. Saturated aqueous ammonium chloride solution (50 mL) was added and the mixture extracted with ethyl acetate (250 mL). The organic phase was separated and washed with water (2×50 mL) then saturated aqueous sodium chloride solution (75 mL). The aqueous phase was dried over anhydrous sodium sulphate, filtered and the filtrate solvents removed in vacuo to leave an orange gum. The crude product was purified by flash chromatography on silica gel (70 g) eluting with dichloromethane/methanol mixture (9:1) to afford the a brown gum which was triturated with diethyl ether:hexane (1:9) mixture, filtered and dried in vacuo to afford the product (250 mg) as a light-brown solid.

LCMS retention time=1.620 minutes; m/z=335.2 [M+H]⁺.

¹H NMR (400 MHz, DMSO-d₆) δ 0.99 (t, 6H, J=7.1 Hz), 2.58 (m, 4H), 2.84 (m, 2H), 4.38 (t, 2H, J=6.0 Hz), 6.64 (s, 1H), 7.47-7.54 (m, 3H), 7.57-7.61 (m, 2H), 8.23 (s, 1H), 12.73 (brs, 1H).

This compound had activity ‘C’ in the fluorescence polarization assay described below.

### Example 10

### 4-(2,4-Dichloro-phenyl)-6-(2-diethylamino-ethoxy)-1H-pyrrolo[2,3-b]pyridine-3-carbonitrile

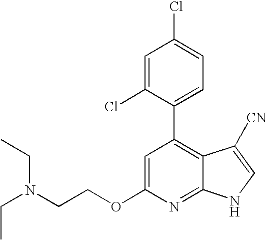

Step 1

### 1-(2,4-dichloro-phenyl)ethanone

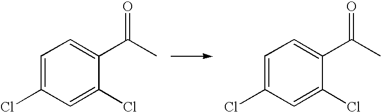

Sodium Hydride (60% dispersion in mineral oil) (4.0 g) was added in portions to a solution of 2,4-dichloroacetophenone (8.8 g) in diethyl carbamate (90 mL) under a nitrogen atmosphere. The mixture was heated to 80° C. for 3 hrs. The mixture was cooled to ambient temperature and then poured into a mixture of water (300 mL) and acetic acid (10 mL). The mixture was extracted with diethyl ether (3×100 mL) and the combined organic extracts were washed with water (2×100 mL), then saturated sodium chloride solution (100 mL). The organic phase was dried over anhydrous sodium sulphate, filtered and the filtrate solvents removed in vacuo to afford an orange oil which was purified by distillation under reduced pressure (1 mm Hg) to afford the product (9.88 g) as a pale yellow oil.

Step 2

### 1-Benzhydryl-6-hydroxy-4-(2,4-dichlorophenyl)-1H-pyrrolo[2,3-b]pyridine-3-carbonitrile

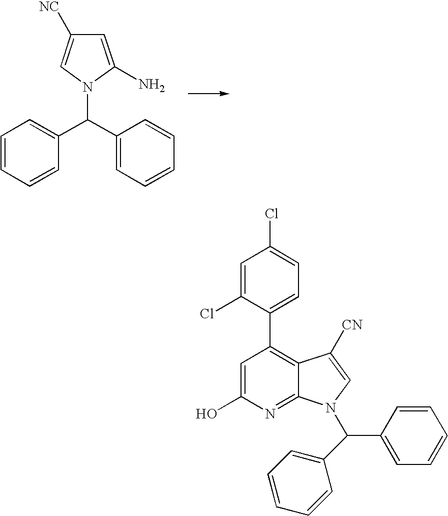

This compound was made utilizing the method of example 9 step 2 using 5-Amino-1-benzhydryl-1H-pyrrole-3-carbonitrile and 1-(2,4-dichloro-phenyl)ethanone.

LCMS retention time=2.733 minutes; m/z=470.1, 472.1 [M+H]⁺.

Step 3

### 1-Benzhydryl-4-(2,4-dichlorophenyl)-6-(2-diethylamino-ethoxy)-1H-pyrrolo[2,3-b]pyridine-3-carbonitrile

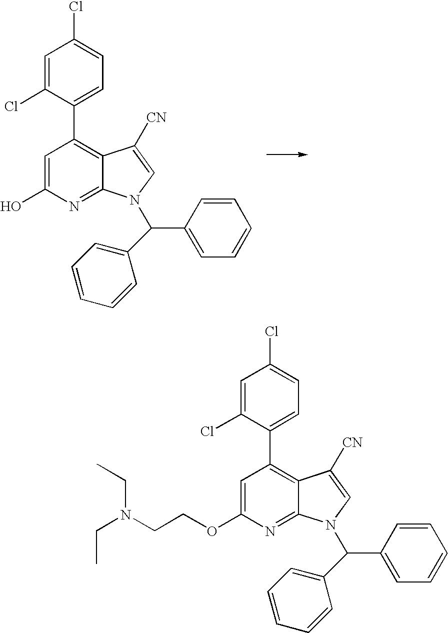

This compound was made by way of the methods utilized for example 9 step 3.

LCMS retention time=2.219 minutes; m/z=569.2, 571.2 [M+H]⁺.

Step 4

### 4-(2,4-Dichloro-phenyl)-6-(2-diethylamino-ethoxy)-1H-pyrrolo[2,3-b]pyridine-3-carbonitrile

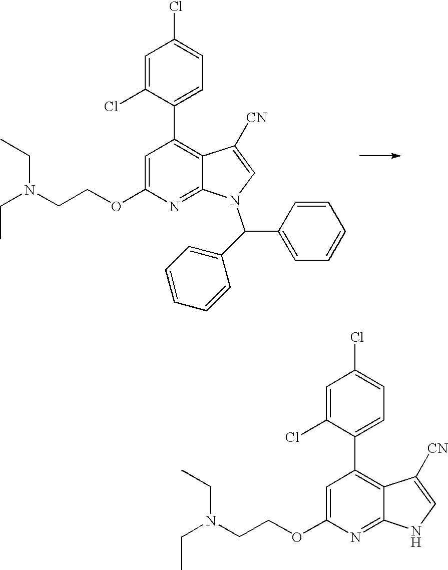

This compound was made by way of the method utilized for example 9 step 4.

LCMS retention time=2.842 minutes; m/z=403.1, 405.1 [M+H]⁺.

¹H NMR (400 MHz, DMSO-d₆) δ 0.99 (t, 6H, J=7.4 Hz), 2.61 (q, 4H, J=7.4 Hz), 2.86 (t, 2H, J=6.1 Hz), 4.39 (t, 2H, J=6.1 Hz), 6.62 (s, 1H), 7.49 (d, 1H, J=8.3 Hz), 7.56 (dd, 1H, J=8.3, 2.0 Hz), 7.80 (d, 1H, J=2.0 Hz), 8.19 (s, 1H), 8.26 (s, 1H)

This compound had activity ‘B’ in the fluorescence polarization assay described below.

### Example 11

### 4-(2,4-Dichloro-phenyl)-6-(3-piperidin-1-yl-propoxy)-1H-pyrrolo[2,3-b]pyridine-3-carbonitrile

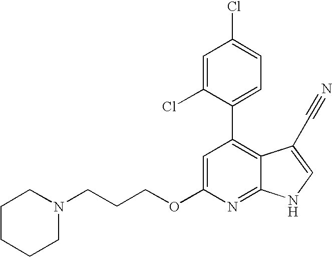

This compound was made by way of the methods utilised for the preparation of the compounds of example 9 and 10. Thus 1-Benzhydryl-4-(2,4-dichlorophenyl)-6-(2-diethylamino-ethoxy)-1H-pyrrolo[2,3-b]pyridine-3-carbonitrile (example 10, step 2) was reacted with 1-(chloropropoxy piperidine hydrochloride then the benzhydryl protecting group removed by the method outlined in example 9 step 4 to afford a crude product which was purified by preparative HPLC to give the title compound (example 11) as a light brown solid.

LCMS retention time=1.895 minutes; m/z=429.3, 431.1 [M+H]⁺.

¹H NMR (400 MHz, DMSO-d₆) δ 1.38 (m, 2H), 1.48 (m. 4H), 1.91 (m, 2H), 2.33 (m, 4H), 2.39 (t, 2H, J=7.0 z), 4.33 (t, 2H, J=7.0 Hz), 6.62 (s, 1H), 7.49 (d, 1H, J=8.2 Hz), 7.56 (dd, 1H, J=8.2 Hz, 2.0 Hz), 7.79 (d, 1H, J=2.0 Hz), 8.18 (s, 1H), 12.70 (brs, 1H).

This compound had activity ‘B’ in the fluorescence polarization assay described below.

### Example 12

### 4-(3-Hydroxy-phenyl)-1H-pyrrolo[2,3-b]pyridine-3-carbonitrile

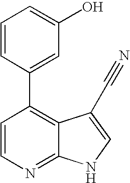

Step 1

### 4-iodo-1H-pyrrolo[2,3-b]pyridine-3-carbonitrile

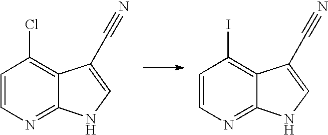

Acetyl chloride (1.5 mL) was added to a suspension of 4-Chloro-1H-pyrrolo[2,3-b]pyridine-3-carbonitrile (1.8 g) and sodium Iodide (7.5 g) in acetonitrile (40 mL) and the suspension was heated to 80° C. for 18 hrs. Ethyl acetate (400 mL) was added and the organic phase was washed sequentially with water (100 mL) 10% (w/v) aqueous sodium thiosulphate solution (100 mL), water (2×100 mL) and saturated aqueous sodium chloride solution (100 mL). The organic phase was dried over sodium sulphate and filtered and filtrate solvents removed in vacuo to give a yellow solid, which was suspended in methanolic ammonia (2M, 35 mL) and stirred for 30 minutes. Methanol was removed in vacuo and the residual solid was triturated with diethyl ether to afford product (2.4 g) as a brown powder.

Step 2

### 4-iodo-1-(2-trimethylsilylanyl-ethoxymethyl)-1H-pyrrolo[2,3-b]pyridine-3-carbonitrile

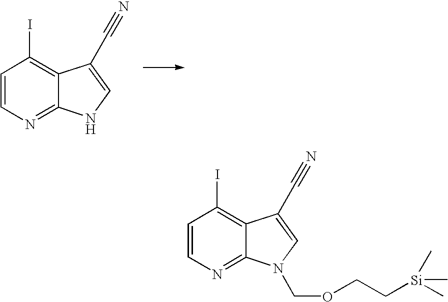

Sodium hydride (400 mg) was added to a suspension of the azaindole (1.5 g) in THF (40 mL) at 0° C. under a nitrogen atmosphere. The mixture was stirred for 90 mins at ambient temperature. The reaction mixture was recooled to 0° C. and 2-(trimethylsilyl)-ethoxy methyl chloride (1.5 mL) was added and the solution was stirred for 2 hours at. Ethyl acetate was added and the organic phase was washed with water (2×100 mL) and saturated aqueous sodium chloride solution (100 mL). The organic phase was dried over sodium sulphate, filtered and the filtrate solvents removed in vacuo to afford a yellow solid. The crude product was purified by flash chromatography eluting with ethyl acetate:hexane mixture (1:9) to afford the product (1.7 g) as a colorless solid.

Step 3

### 4-(3-hydroxy-phenyl)-1-(2-trimethylsilylanyl-ethoxymethyl)-1H-pyrrolo[2,3-b]pyridine-3-carbonitrile

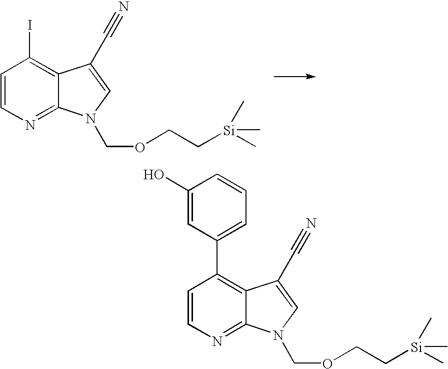

Potassium phosphate (5.1 g) was added to a suspension of 3-hydroxyphenyl boronic acid (1.1 g) and 4-iodo-1-(2-trimethylsilylanyl-ethoxymethyl)-1H-pyrrolo[2,3-b]pyridine-3-carbonitrile (1.6 g) in 1,4-dioxane (20 mL) under a nitrogen atmosphere. Dichloro-bis-(triphenylphosphine) palladium II (105 mg) was added and the reaction mixture was heated to 95° C. for 18 hr. The suspension was allowed to cool and ethyl acetate (150 mL) was added and the mixture was washed sequentially with saturated ammonium chloride solution (50 mL), water (2×50 mL) and saturated aqueous sodium chloride solution (50 mL). The organic phase was dried over sodium sulphate, filtered and filtrate solvents removed in vacuo to afford a brown gum. The crude product was purified by flash chromatography eluting with ethyl acetate:hexane mixture (1:2) to afford the product (1.4 g) as a colorless solid.

Step 4

### 4-(3-Hydroxy-phenyl)-1H-pyrrolo[2,3-b]pyridine-3-carbonitrile

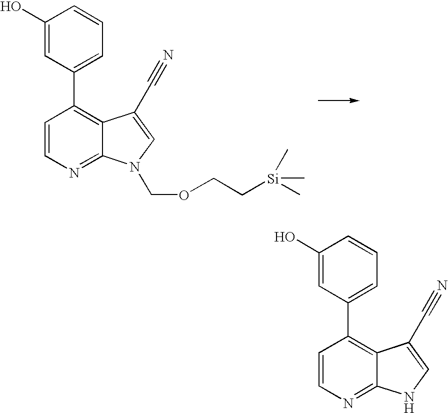

The compound was made by the method utilized for example 4 step 3.

LCMS retention time=1.810 minutes; m/z=236.1 [M+H]⁺.

¹H NMR (400 MHz, DMSO-d₆) δ 6.88 (dd, 1H, J=7.4, 2.5 Hz), 6.91 (t, 1H, J=2.5 Hz), 7.00 (d, 1H, J=7.8 Hz), 7.20 (d, 1H, J=4.8 Hz), 7.31 (t, 1H, J=7.8 Hz), 8.41 (d, 1H, J=4.8 Hz), 8.50 (s, 1H), 9.65 (brs, 1H), 12.96 (brs, 1H).

This compound had activity ‘B’ in the fluorescence polarization assay described below.

### Example 13

### 4-[3-(2-Diethylamino-ethoxy)-phenyl]-1H-pyrrolo[2,3-b]pyridine-3-carbonitrile

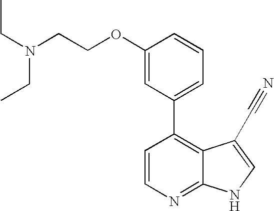

Step 1

### 4-[3-(2-Diethylamino-ethoxy)-phenyl]-1-(2-trimethylsilanyl-ethoxymethyl)-1H-pyrrolo[2,3-b]pyridine-3-carbonitrile

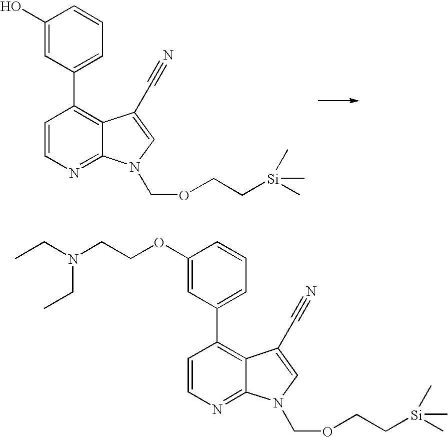

A solution of diisopropylazodicarboxylate (0.15 mL) in THF (2.5 mL) was added to a solution of 4-(3-hydroxy-phenyl)-1-(2-trimethylsilylanyl-ethoxymethyl)-1H-pyrrolo[2,3-b]pyridine-3-carbonitrile (180 mg) and triphenyl phosphine (200 mg) in THF (10 mL) under a nitrogen atmosphere. A solution of 2-diethylamino ethanol (0.10 mL) in THF (2.5 mL) was then added and the mixture stirred at ambient temperature for 18 hr. The product was purified by ion exchange chromatography (SCX-II), followed by flash chromatography on silica gel (20 g) eluting with ethyl acetate to give the product (190 mg) as a pale brown gum.

Step 2

### 4-[3-(2-Diethylamino-ethoxy)-phenyl]-1H-pyrrolo[2,3-b]pyridine-3-carbonitrile

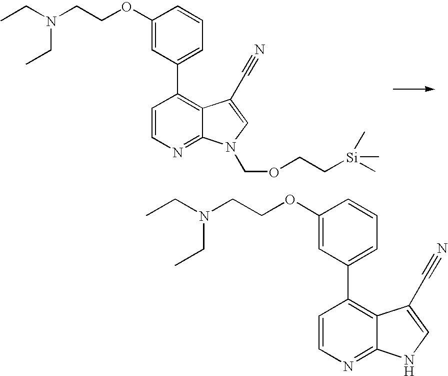

The compound was made by the method utilized for example 4 step 3.

LCMS retention time=1.56 minutes; m/z=335.2 [M+H]⁺.

¹H NMR (400 MHz, DMSO-d₆) δ 0.97 (t, 6H, J=7.1 Hz), 2.55 (q, 4H, J=7.1 Hz)), 2.81 (t, 2H, J=6.1 Hz)), 4.13 (t, 2H, J=6.1 Hz), 7.04 (m, 1H), 7.15 (m, 1H), 7.17 (m, 1H), 7.27 (d, 1H, J=4.8 Hz), 7.42 (t, 1H, J=7.8 Hz), 8.43 (d, 1H, J=4.8 Hz), 8.52 (s, 1H), 12.95 (brs, 1H).

This compound had activity ‘B’ in the fluorescence polarization assay described below.

### Example 14

### 6-Chloro-4-(3-hydroxy-phenyl)-1H-pyrrolo[2,3-b]pyridine-3-carbonitrile

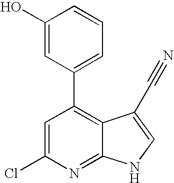

Step 1

### 4-(3-hydroxy-phenyl)-7-oxo-1H-pyrrolo[2,3-b]pyridine-3-carbonitrile methane sulphonate

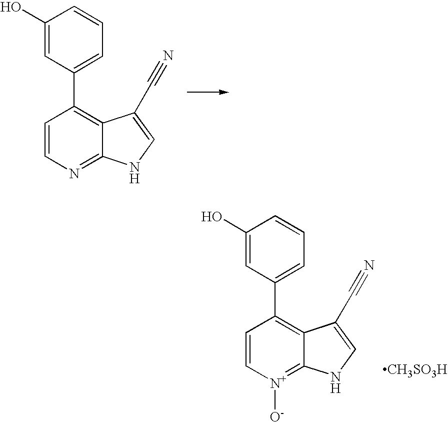

4-(3-Hydroxy-phenyl)-1H-pyrrolo[2,3-b]pyridine-3-carbonitrile (850 mg) was added to a solution of meta-chloroperoxybenzoic acid (ca 77%; 1.6 g) in dichloromethane (40 mL). The reaction mixture was stirred for ca. 100 hrs affording a yellow suspension. Methane sulphonic acid (0.5 mL) was added affording a yellow solution. Diethyl ether was added and the mixture stirred to afford a yellow suspension, which was filtered, washed with ether and dried in vacuo to give the product (560 mg) as a yellow powder.

Step 2

### 6-Chloro-4-(3-hydroxy-phenyl)-1H-pyrrolo[2,3-b]pyridine-3-carbonitrile

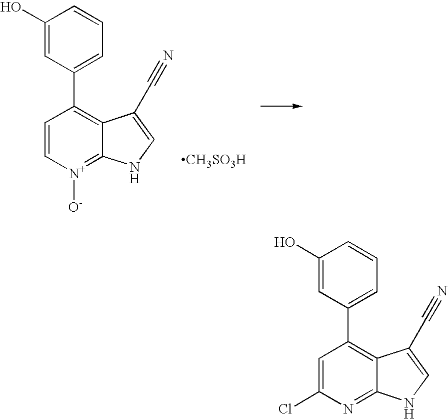

Methane sulfonyl chloride (1.0 mL) was added to a solution of 4-(3-hydroxy-phenyl)-7-oxo-1H-pyrrolo[2,3-b]pyridine-3-carbonitrile methane sulphonate (550 mg) in DMF (10 mL) and the mixture was heated at 80° C. for 90 mins. The resulting solution was allowed to cool to ambient temperature and then poured into water (100 mL) and the suspension was made basic by addition of aqueous ammonia, and stirred for 10 min. The solids were collected by filtration and then purified by flash chromatography on silica gel, eluting with dichloromethane/methanol (19:1) to give a pale brown solid (300 mg).

LCMS retention time=2.09 minutes; m/z=270 [M+H]⁺.

¹H NMR (400 MHz, DMSO-d₆) δ 6.91 (dd, 1H, J=8.1, 2.4 Hz), 6.97 (t, 1H, J=2.4 Hz), 7.02 (d, 1H, J=8.1 Hz), 7.28 (s, 1H), 7.33 (t, 1H, J=7.8 Hz), 8.54 (s, 1H), 9.72 (s, 1H), 13.17 (brs, 1H).

This compound had activity ‘A’ in the fluorescence polarization assay described below.

### Example 15

### 6-methoxy-4-(-phenyl)-1H-pyrrolo[2,3-b]pyridine-3-carbonitrile

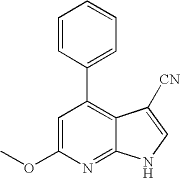

This compound was made by way of the methods utilized for example 9, using dimethyl sulphate to alkylate 1-Benzhydryl-6-hydroxy-4-phenyl-1H-pyrrolo[2,3-b]pyridine-3-carbonitrile (example 9 step 3).

LCMS retention time=2.34 minutes; m/z=250.1 [M+H]⁺.

¹H NMR (400 MHz, DMSO-d₆) δ 3.93 (s, 1H), 6.68 (s, 1H), 7.49-7.62 (m, 3H), 7.58-7.62 (m, 2H), 8.24 (d, 1H, J=3.0 Hz), 12.77 (brs, 1H).

This compound had activity ‘A’ in the fluorescence polarization assay described below.

Fluorescence Polarization Assay

Fluorescence polarization {also known as fluorescence anisotropy} measures the rotation of a fluorescing species in solution, where the larger molecule the more polarized the fluorescence emission. When the fluorophore is excited with polarized light, the emitted light is also polarized. The molecular size is proportional to the polarization of the fluorescence emission.

The fluoroscein-labelled probe—

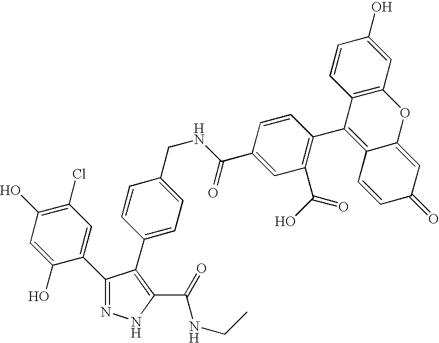

binds to HSP90 {full-length human, full-length yeast or N-terminal domain HSP90} and the anisotropy {rotation of the probe:protein complex} is measured.

Test compound is added to the assay plate, left to equilibrate and the anisotropy measured again. Any change in anisotropy is due to competitive binding of compound to HSP90, thereby releasing probe.

Materials

Chemicals are of the highest purity commercially available and all aqueous solutions are made up in AR water.
1) Costar 96-well black assay plate #3915
2) Assay buffer of (a)100 mM Tris pH7.4; (b) 20 mM KCl; (c) 6 mM MgCl₂. Stored at room temperature.
3) BSA (bovine serum albumen) 10 mg/ml (New England Biolabs # B9001S)
4) 20 mM probe in 100% DMSO stock concentration. Stored in the dark at RT. Working concentration is 200 nM diluted in AR water and stored at 4° C. Final concentration in assay 80 nM.
5) E. coli expressed human full-length HSP90 protein, purified >95% (see, e.g., Panaretou et al., 1998) and stored in 50 μL aliquots at −80° C.

Protocol
1) Add 100 μl 1× buffer to wells 11A and 12A (=FP BLNK)
2) Prepare assay mix—all reagents are kept on ice with a lid on the bucket as the probe is light-sensitive.

| | i. Final Concⁿ | | | |
|---|---|---|---|---|
| 1x Hsp90 FP Buffer | 10 | ml | 1x | |
| BSA 10 mg/ml (NEB) | 5.0 | μl | 5 | μg/ml |
| Probe 200 μM | 4.0 | μl | 80 | nM |
| Human full-length Hsp90 | 6.25 | μl | 200 | nM |

3) Aliquot 100 μl assay mix to all other wells
4) Seal plate and leave in dark at room temp for 20 minutes to equilibrate

Compound Dilution Plate—1×3 dilution series
1) In a clear 96-well v-bottom plate—{# VWR 007/008/257} add 10 μl 100% DMSO to wells B1 to H11
2) To wells A1 to A11 add 17.5 μl 100% DMSO
3) Add 2.5 μl cpd to A1. This gives 2.5 mM {50×} stock cpd—assuming cpds 20 mM.
4) Repeat for wells A2 to A10. Control in columns 11 and 12.
5) Transfer 5 μl from row A to row B—not column 12. Mix well.
6) Transfer 5 μl from row B to row C. Mix well.
7) Repeat to row G.
8) Do not add any compound to row H—this is the 0 row.
9) This produces a 1×3 dilution series from 50 μM to 0.07 μM.
10) In well B12 prepare 20 μl of 100 μM standard compound.
11) After first incubation the assay plate is read on a Fusion™ α-FP plate reader (Packard BioScience, Pangbourne, Berkshire, UK).
12) After the first read, 2 μl of diluted compound is added to each well for columns 1 to 10. In column 11 {provides standard curve} only add compound B11-H11. Add 2 μl of 100 mM standard cpd to wells B12-H12 {is positive control}
13) The Z′ factor is calculated from zero controls and positive wells. It typically gives a value of 0.7-0.9.

The compounds tested in the above assay were assigned to one of two activity ranges, namely

A: FP IC₅₀<1.0 μM

B: FP IC₅₀>1.0 μM<10 μM

C: FP IC₅₀>10.0 μM,

and those assignments are reported above.

A growth inhibition assay was also employed for the evaluation of candidate HSP90 inhibitors:

Assessment of Cytotoxicity by Sulforhodamine B (SRB) Assay: Calculation of 50% Inhibitory Concentration (IC₅₀).

Day 1
1) Determine cell number by haemocytometer.
2) Using an 8 channel multipipettor, add 160 μl of the cell suspension (3600 cells/well or 2×10⁴ cells/ml) to each well of a 96-well microtitre plate.
3) Incubate overnight at 37° C. in a CO₂ incubator.

Day 2
4) Stock solutions of drugs are prepared, and serial dilutions of each drug are performed in medium to give final concentrations in wells.
5) Using a multipipettor, 40 μl of drug (at 5× final concentration) is added to quadruplicate wells.
6) Control wells are at either side of the 96 well plates, where 40 μl of medium is added.
7) Incubate plates in CO₂ incubator for 4 days (48 hours).

Day 6
8) Tip off medium into sink and immerse plate slowly into 10% ice cold trichloroacetic acid (TCA). Leave for about 30 mins on ice.
9) Wash plates three times in tap water by immersing the plates into baths of tap water and tipping it off.
10) Dry in incubator.
11) Add 100 μl of 0.4% SRB in 1% acetic acid to each well (except the last row (right hand) of the 96 well plate, this is the 0% control, ie no drug, no stain. The first row will be the 100% control with no drug, but with stain). Leave for 15 mins.
12) Wash off unbound SRB stain with four washes of 1% acetic acid.
13) Dry plates in incubator.
14) Solubilise SRB using 100 μl of 10 mM Tris base and put plates on plate shaker for 5 mins.
15) Determine absorbance at 540 nm using a plate reader. Calculate mean absorbance for quadruplicate wells and express as a percentage of value for control, untreated wells.
16) Plot % absorbance values versus log drug concentration and determine the IC₅₀.

The compounds tested in the above assay were assigned to one of two activity ranges, namely A=IC₅₀<50 μM; B=IC₅₀>50 μM. By way of example the compound of example 5 had activity in the “A” range”.

### REFERENCES

A number of publications are cited above in order to more fully describe and disclose the invention and the state of the art to which the invention pertains. Full citations for these references are provided below. Each of these references is incorporated herein by reference in its entirety into the present disclosure.

Reviews for detailed background and drug discovery:
Isaacs J S. 2005 Heat-shock protein 90 inhibitors in antineoplastic therapy: is it all wrapped up?, Expert Opin. Investig. Drugs 14, 569-589.
Maloney A and Workman P. 2002 Hsp90 as a new therapeutic target for cancer therapy: the story unfolds, Expert Opin. Biol. Ther. 2, 3-24.
Whitesell L and Lindquist S L. 2005 Hsp90 and the chaperoning of cancer, Nature Rev. Cancer 5, 761-772.
Janin Y, Heat Shock Protein 90 Inhibitors. A Text Book Example of Medicinal Chemistry? J. Med. Chem. 2005, 48, 7503.
Aoyagi S and Archer T K. 2005 Modulating molecular chaperone Hsp90 functions through reversible acetylation, Trends Cell Biol. 15, 565-7.
Argon Y and Simen B B. 1999 Grp94, an ER chaperone with protein and peptide binding properties, Semin. Cell Dev. Biol. 10, 495-505.
Bijlmakers M-J J E, Marsh M. 2000 Hsp90 is essential for the synthesis and subsequent membrane association, but not the maintenance, of the Src-kinase p56lck, Molecular Biology of the Cell 11, 1585-1595.
Bucci M; Roviezzo F; Cicala C; Sessa W C, Cirino G. 2000 Geldanamycin, an inhibitor of heat shock protein 90 (Hsp90) mediated signal transduction has anti-inflammatory effects and interacts with glucocorticoid receptor in vivo, Brit. J. Pharmacol. 131, 13-16.
Chen C-F, Chen Y, Dai K D, Chen P-L, Riley D J and Lee W-H. 1996 A new member of the hsp90 family of molecular chaperones interacts with the retinoblastoma protein during mitosis and after heat shock, Mol. Cell. Biol. 16, 4691-4699.
Chiosis G, Timaul M N, Lucas B, Munster P N, Zheng F F, Sepp-Lozenzino L and Rosen N. 2001 A small molecule designed to bind to the adenine nucleotide pocket of Hsp90 causes Her2 degradation and the growth arrest and differentiation of breast cancer cells, Chem. Biol. 8, 289-299.
Conroy S E and Latchman D S. 1996 Do heat shock proteins have a role in breast cancer?, Brit. J. Cancer 74, 717-721.
Dymock B, Barril X, Beswick M, Collier A, Davies N, Drysdale M, Fink A, Fromont C, Hubbard R E, Massey A, Surgenor A, Wright L. Adenine Derived inhibitors of the molecular chaperone HSP90-SAR explained through multiple X-ray structures. Bioorg. Med. Chem. Lett., 2004, 14, 325.
da Rocha Dias S, Friedlos F, Light Y, Springer C, Workman P and Marais R. 2005 Activated B-Raf is an Hsp90 client protein that is targeted by the anticancer drug 17-Allylamino-17-demethoxygeldanamycin, Cancer Res. 65, 10686-10691.
Felts S J, Owen B A L, Nguyen P, Trepel J, Donner D B and Toft D O. 2000 The Hsp90-related protein TRAP1 is a mitochondrial protein with distinct functional properties, J. Biol. Chem. 5, 3305-3312.
Fuller W, Cuthbert A W. 2000 Post-translational disruption of the delta F508 cystic fibrosis transmembrane conductance regulator (CFTR)-molecular Chaperone complex with geldanamycin stabilizes delta F508 CFTR in the rabbit reticulocyte lysate, J. Biol. Chem. 275(48), 37462-37468.
Hickey E, Brandon S E, Smale G, Lloyd D and Weber L A. 1999 Sequence and regulation of a gene encoding a human 89-kilodalton heat shock protein, Mol. Cell. Biol. 9, 2615-2626.
Hoang A T, Huang J, Rudra-Gonguly N, Zheng J, Powell W C, Rabindron S K, Wu C and Roy-Burman P. 2000 A novel association between the human heat shock transcription factor I (HSF1) and prostate adenocarcinoma, Am. J. Pathol. 156, 857-864.
Hostein I, Robertson D, Di Stefano F, Workman P and Clarke P A. 2001 Inhibition of signal transduction by the Hsp90 inhibitor 17-allylamino-17-demethoxygeldanamycin results in cytostasis and apoptosis, Cancer Res. 61, 4003-4009.
Hur E, Kim H-H, Choi S M, Kim J H, Yim S, Kwon H J, Choi Y, Kim D K, Lee M-O, Park H. 2002 Reduction of hypoxia-induced transcription through the repression of hypoxia-inducible factor-1□/aryl hydrocarbon receptor nuclear translocator DNA binding by the 90-kDa heat-shock protein inhibitor radicicol, Mol. Pharmacol. 62(5), 975-982.
Hutter et al. 1996 Circulation 94, 1408.
Jameel A, Skilton R A, Campbell T A, Chander S K, Coombes R C and Luqmani Y A. 1992 Clinical and biological significance of Hsp89a in human breast cancer, Int. J. Cancer 50, 409-415.
Jolly C and Morimoto R I. 2000 Role of the heat shock response and molecular chaperones in oncogenesis and cell death, J. Natl. Cancer Inst. 92, 1564-1572.
Kamal A, Thao L, Sensintaffar J, Zhang L, Boehm M F, Fritz L C and Burrows F J. 2003 A high-affinity conformation of Hsp90 confers tumour selectivity on Hsp90 inhibitors, Nature 425, 407-410.
Kasibhatla S R, Hong K, Zhang L, Biamonte M A, Boehm M F, Shi J, Fan J. PCT Int Appl. WO 2003037860.
Kawanishi K, Shiozaki H, Doki Y, Sakita I, Inoue M, Yano M, Tsujinata T, Shamma A and Monden M. 1999 Prognostic significance of heat shock proteins 27 and 70 in patients with squamous cell carcinoma of the esophagus, Cancer 85, 1649-1657.
Kelland L R, Abel G, McKeage M J, Jones M, Goddard P M, Valenti M, Murrer B A and Harrap K R. 1993 Preclinical antitumour evaluation of bis-acetalo-amino-dichloro-cyclohexylamine platinum (IV): an orally active platinum drug, Cancer Research 53, 2581-2586.
Kelland L R, Sharp S Y, Rogers P M, Myers T G and Workman P. 1999 DT-diaphorase expression and tumor cell sensitivity to 17-allylamino, 17-demethoxygeldanamycin, an inhibitor of heat shock protein 90, J. Natl. Cancer Inst. 91, 1940-1949.
Kovacs J J, Murphy P J M, Gaillard S, Zhao X, Wu J-T, Nicchitta C V, Yoshida M, Toft D O, Pratt W B and Yao T-P. 2005 HDAC6 regulates Hsp90 acetylation and chaperone-dependent activation of the glucocorticoid receptor, Mol. Cell 18, 601-607.
Kurebayashi J, Otsuki T, Kurosumi M, Soga S, Akinaga S, Sonoo, H. 2001 A radicicol derivative, KF58333, inhibits expression of hypoxia-inducible factor-1□ and vascular endothelial growth factor, angiogenesis and growth of human breast cancer xenografts, Jap. J. Cancer Res. 92(12), 1342-1351.
Kwon H J, Yoshida M, Abe K, Horinouchi S and Bepple T. 1992 Radicicol, an agent inducing the reversal of transformed phentoype of src-transformed fibroblasts, Biosci., Biotechnol., Biochem., 56, 538-539.
Lebeau J, Le Cholony C, Prosperi M T and Goubin G. 1991 Constitutive overexpression of 89 kDa heat shock protein gene in the HBL100 mammary cell line converted to a tumorigenic phenotype by the EJ/T24 Harvey-ras oncogene, Oncogene 6, 1125-1132.
Llauger L, He, H, Kim J, Aguirre J, Rosen N, Peters U; Davies P, Chiosis G, J. Med. Chem. 2005, 48, 2892
Marcu M G, Chadli A, Bouhouche I, Catelli M and Neckers L. 2000a The heat shock protein 90 antagonist novobiocin interacts with a previously unrecognized ATP-binding domain in the carboxyl terminus of the chaperone, J. Biol. Chem. 275, 37181-37186.
Marcu M G, Schulte T W and Neckers L. 2000b Novobiocin and related coumarins and depletion of heat shock protein 90-dependent signaling proteins, J. Natl. Cancer Inst. 92, 242-248.
Martin K J, Kritzman B M, Price L M, Koh B, Kwan C P, Zhang X, MacKay A, O'Hare M J, Kaelin C M, Mutter G L, Pardee A B and Sager R. 2000 Linking gene expression patterns to therapeutic groups in breast cancer, Cancer Res. 60, 2232-2238.
Neckers L, Schulte T W and Momnaaugh E. 1999 Geldanamycin as a potential anticancer agent: its molecular target and biochemical activity, Invest. New Drugs 17, 361-373.
Page J, Heath J, Fulton R, Yalkowsky E, Tabibi E, Tomaszewski J, Smith A and Rodman L. 1997 Comparison of geldanamycin (NSC-122750) and 17-allylaminogeldanamycin (NSC-330507D) toxicity in rats, Proc. Am. Assoc. Cancer Res. 38, 308.
Panaretou B, Prodromou C, Roe S M, O'Brien R, Ladbury J E, Piper P W and Pearl L H. 1998 ATP binding and hydrolysis are essential to the function of the Hsp90 molecular chaperone in vivo, EMBO J. 17, 4829-4836.
Plumier et al. 1997 Cell. Stress Chap., 2, 162
Pratt W B. 1997 The role of the Hsp90-based chaperone system in signal transduction by nuclear receptors and receptors signalling via MAP kinase, Annu. Rev. Pharmacol. Toxicol. 37, 297-326.
Prodromou C and Pearl L H. 2000a Structure and in vivo function of Hsp90, Curr. Opin. Struct. Biol. 10, 46-51.
Prodromou C, Roe SM, O'Brien R, Ladbury J E, Piper P W and Pearl L H. 1997 Identification and structural characterization of the ATP/ADP-binding site in the Hsp90 molecular chaperone, Cell 90, 65-75.
Prodromou C, Panaretou B, Chohan S, Siligardi G, O'Brien R, Ladbury J E, Roe S M, Piper P W and Pearl L H. 2000b The ATPase cycle of Hsp90 drives a molecular ‘clamp’ via transient dimerization of the N-terminal domains, EMBO J. 19, 4383-4392.
Rajder et al. 2000 Ann. Neurol. 47, 782.
Roe S M, Prodromou C, O'Brien R, Ladbury J E, Piper P W and Pearl L H. 1999 Structural basis for inhibition of the Hsp90 molecular chaperone by the antitumour antibiotics radicicol and geldanamycin, J. Med. Chem. 42, 260-266.
Rutherford S L and Lindquist S. 1998 Hsp90 as a capacitor for morphological evolution. Nature 396, 336-342.
Schulte T W, Akinaga S, Murakata T, Agatsuma T, Sugimoto S, Nakano H, Lee Y S, Simen B B, Argon Y, Felts S, Toft D O, Neckers L M and Sharma S V. 1999 Interaction of radicicol with members of the heat shock protein 90 family of molecular chaperones, Mol. Endocrinology. 13, 1435-1448.
Schulte T W, Akinaga S, Soga S, Sullivan W, Sensgard B, Toft D and Neckers L M. 1998 Antibiotic radicicol binds to the N-terminal domain of Hsp90 and shares important biologic activities with geldanamcyin, Cell Stress and Chaperones 3, 100-108.
Schulte T W and Neckers L M. 1998 The benzoquinone ansamycin 17-allylamino-17-deemthoxygeldanamcyin binds to Hsp90 and shares important biologic activities with geldanamycin, Cancer Chemother. Pharmacol. 42, 273-279.
Siligardi G, Hu B, Panaretou B, Piper P W, Pearl L H and Prodromou C. 2004 Co-chaperone regulation of conformational switching in the Hsp90 ATPase cycle, J. Biol. Chem. 279, 51989-51998.
Sittler et al. 2001 Hum. Mol. Genet. 10, 1307.
Smith D F. 2001 Chaperones in signal transduction, in: Molecular chaperones in the cell (P Lund, ed.; Oxford University Press, Oxford and NY), 165-178.
Smith D F, Whitesell L and Katsanis E. 1998 Molecular chaperones: Biology and prospects for pharmacological intervention, Pharmacological Reviews 50, 493-513.
Song H Y, Dunbar J D, Zhang Y X, Guo D and Donner D B. 1995 Identification of a protein with homology to hsp90 that binds the type 1 tumour necrosis factor receptor, J. Biol. Chem. 270, 3574-3581.
Stebbins C E, Russo A, Schneider C, Rosen N, Hartl F U and Pavletich N P. 1997 Crystal structure of an Hsp90-geldanamcyin complex: targeting of a protein chaperone by an antitumor agent, Cell 89, 239-250.
Supko J G, Hickman R L, Grever M R and Malspeis L. 1995 Preclinical pharmacologic evaluation of geldanamycin as an antitumour agent, Cancer Chemother. Pharmacol. 36, 305-315.
Tratzelt et al. 1995 Proc. Nat. Acad. Sci. 92, 2944.
Trost et al. 1998 J. Clin. Invest. 101, 855.
Tytell M and Hooper P L. 2001 Heat shock proteins: new keys to the development of cytoprotective therapies, Emerging Therapeutic Targets 5, 267-287.
Uehara U, Hori M, Takeuchi T and Umezawa H. 1986 Phenotypic change from transformed to normal induced by benzoquinoid ansamycins accompanies inactivation of p60src in rat kidney cells infected with Rous sarcoma virus, Mol. Cell. Biol. 6, 2198-2206.
Waxman, Lloyd H. Inhibiting hepatitis C virus processing and replication. (Merck & Co., Inc., USA). PCT Int. Appl. (2002), WO 0207761
Winklhofer et al. 2001 J. Biol. Chem. 276, 45160.
Whitesell L, Mimnaugh E G, De Costa B, Myers C E and Neckers L M. 1994 Inhibition of heat shock protein Hsp90-pp 60v-src heteroprotein complex formation by benzoquinone ansamycins: essential role for stress proteins in oncogenic transformation, Proc. Natl. Acad. Sci. USA. 91, 8324-8328.
Yorgin et al. 2000 Effects of geldanamycin, a heat-shock protein 90-binding agent, on T cell function and T cell nonreceptor protein tyrosine kinases, J. Immunol. 164(6), 2915-2923.
Young J C, Moarefi I and Hartl F U. 2001 Hsp90: a specialized but essential protein-folding tool, J. Cell. Biol. 154, 267-273.
Zhao J F, Nakano H and Sharma S. 1995 Suppression of RAS and MOS transformation by radicicol, Oncogene 11, 161-173.

1. A compound of formula (I), or a salt, N-oxide, hydrate, or solvate thereof: 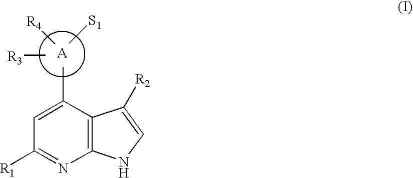
wherein
ring A is an aryl or heteroaryl ring or ring system;
R₁ is hydrogen, fluoro, chloro, bromo, or a radical of formula (1A):
—X-Alk¹-(Z)ₘ-(Alk²)ₙ-Q (IA)
wherein
X is a bond, —O—, —S— —S(O)—, —SO₂—, or —NH—,
Z is —O—, —S—, —(C═O)—, —(C═S)—, —S(O)—, —SO₂—, —NR^{A}—, or, in either orientation —C(═O)O—, —C(═O)NR^{A}—, —C(═S)NR^{A}—, —SO₂NR^{A}—, —NR^{A}C(═O)—, or —NR^{A}SO₂— wherein R^{A} is hydrogen or C₁-C₆ alkyl in which one or more hydrogens is optionally substituted by fluorine;
Alk¹ and Alk² are optionally substituted divalent C₁-C₃ alkylene or C₂-C₃ alkenylene radicals,
m and n are independently 0 or 1, and
Q is hydrogen or an optionally substituted carbocyclic or heterocyclic radical;
R₂ is cyano (—CN), fluoro, chloro, bromo, methyl, ethyl, —OH, —CH₂OH, —C(═O)NH₂, —C(═O)H, —C(═O)CH₃, or —NH₂;
R₃ and R₄ are independently selected from hydrogen, fluoro, chloro, bromo, cyano (—CN), C₁-C₃alkyl optionally substituted with one or more fluorine substituents, C₁-C₃alkoxy optionally substituted with one or more fluorine substituents, —CH═CH₂, —C≡CH, cyclopropyl and —NH₂, or R₃ and R₄ together represent methylenedioxy (—OCH₂O—) or ethylenedioxy (—OCH₂CH₂O—) in either of which one or more hydrogens are optionally replaced by fluorine;
S₁ is hydrogen, or a substituent selected from fluoro, chloro, bromo, cyano (—CN), C₁-C₃alkyl optionally substituted with one or more fluorine substituents, C₁-C₃alkoxy optionally substituted with one or more fluorine substituents, —CH═CH₂, —C≡CH, cyclopropyl and —NH₂, or S₁ and R₃, or S₁ and R₄, together represent methylenedioxy (—OCH₂O—) or ethylenedioxy ((—OCH₂ CH₂O—) in either of which one or more hydrogens are optionally replaced by fluorine; or SI is a radical of formula (IB):
-(Alk³)ₚ-(Z¹)_{q}-(Alk⁴)ᵣ-Q¹ (IB)
wherein
p, q and r are independently 0 or 1;
(a) when p is 0 or 1, and q is 1, and r is 0 or 1:
Z¹ is selected from the group of divalent radicals consisting of (i) —S—, —(C═O)—,
—(C═S)—, —S(O)— and —SO₂— and (ii) —N(R^{A})C(═O)—* wherein the bond marked * is attached to Q¹ and (iii) in either orientation, —C(═O)O—, —C(═S)NR^{A}—, and —SO₂NR^{A}—; and Q¹ is (i) hydrogen or an optional substituent; or (ii) an optionally substituted carbocyclic or heterocyclic radical; or (iii) a radical —CH₂[O(CH₂)_{w}]ₓZ² wherein Z² is H, —OH or —O(C₁-C₃alkyl) wherein x and w are independently 1, 2 or 3; or
(b) when p is 1, and q is 1, and r is 0 or 1:
Z¹ is —O—, and Q¹ is (i) hydrogen or an optional substituent which is not linked to -(Alk³)ₚ-(Z¹)_{q}-(Alk⁴)ᵣ- through a nitrogen atom; or (ii) an optionally substituted carbocyclic radical; or (iii) an optionally substituted heterocyclic ring of 5 or 6 ring atoms which is not linked to -(Alk³)ₚ-(Z¹)_{q}-(Alk⁴)ᵣ- through a ring nitrogen; or (iv) a radical —CH₂[O(CH₂)_{w}]ₓZ² wherein Z² is H, —OH or —O(C₁-C₃alkyl) wherein x and w are independently 1, 2 or 3, or
(c) when p is 1, and q is 1, and r is 0 or 1:
Z¹ is —NR^{A}— or —C(═O)N(R^{A})—* wherein the bond marked * is attached to Q¹ and Q¹ is a radical —CH₂[O(CH₂)_{w}]ₓZ² wherein Z² is H, —OH or —O(C₁-C₃alkyl) wherein x and w are independently 1, 2 or 3, or
(d) when p is 0, and q is 1, and r is 0 or 1:
Z¹ is —O— or —NR^{A}— and Q¹ is (i) hydrogen or an optional substituent which is not linked to -(Alk³)ₚ-(Z¹)_{q}-(Alk⁴)ᵣ- through a nitrogen atom; or (ii) Q¹ and R^{A}, taken together with the nitrogen to which they are attached form an optionally substituted heterocyclic ring of 5 or 6 ring atoms; or (iii) a radical —CH₂[O(CH₂)_{w}]ₓZ² wherein Z² is H, —OH or —O(C₁-C₃alkyl) wherein x and w are independently 1, 2 or 3; or
(e) when p is 0 or 1, q is 0, and r is 0 or 1:
Q¹ is (i) hydrogen or an optional substituent which is not linked to -(Alk³)ₚ-(Z¹)_{q}-(Alk⁴)ᵣ- through a nitrogen atom or (ii) an optionally substituted carbocyclic radical; or (iii) an optionally substituted heterocyclic of 5 or 6 ring atoms which is not linked to -(Alk³)ₚ-(Z¹)_{q}-(Alk⁴)ᵣ- through a ring nitrogen; or (iv) a radical —CH₂[O(CH₂)_{w}]ₓZ² wherein Z² is H, —OH or —O(C₁-C₃alkyl) wherein x and w are independently 1, 2 or 3;
R^{A} is hydrogen or C₁-C₃ alkyl optionally substituted with one or more fluorine substituents; and
Alk³ and Alk⁴ are divalent C₁-C₃ alkylene or C₂-C₃ alkenylene radicals, each optionally substituted by one or two substituents selected from fluoro, chloro, C₁-C₃alkyl optionally substituted with one or more fluorine substituents, C₁-C₃alkoxy optionally substituted with one or more fluorine substituents.

2. A compound as claimed in claim 1 wherein ring A is a phenyl ring.

3. A compound of formula (IC), or a salt, N-oxide, hydrate, or solvate thereof: 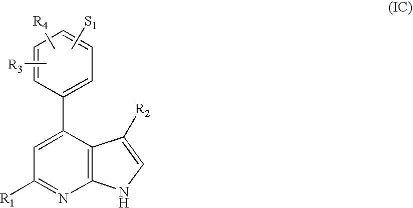
wherein
R₁, R₂, R₃ and R₄ are as defined in claim 1, and
S₁ is hydrogen, or a substituent selected from fluoro, chloro, bromo, cyano (—CN), C₁-C₃alkyl optionally substituted with one or more fluorine substituents, C₁-C₃alkoxy optionally substituted with one or more fluorine substituents, —CH═CH₂, —C≡CH, cyclopropyl and —NH₂, or S₁ and R₃, or S₁ and R₄, together represent methylenedioxy (—OCH₂O—) or ethylenedioxy ((—OCH₂CH₂O—) in either of which one or more hydrogens are optionally replaced by fluorine;
or S₁ is a radical of formula (IB):
-(Alk³)ₚ-(Z¹)_{q}-(Alk⁴)ᵣ-Q¹ (IB)
wherein
p, q and r are independently 0 or 1;
Z¹ is —O—, —S—, —(C═O)—, —(C═S)—, —S(O)—, —SO₂—, —NR^{A}—, or, in either orientation,
—C(═O)N(R^{A})— or —SO₂NR^{A}—;
Q¹ is (i) hydrogen or an optional substituent; or (ii) an optionally substituted carbocyclic or heterocyclic radical; or (iii) a radical CH₂[O(CH₂)_{w}]ₓZ² wherein Z² is H, —OH or —O(C₁-C₃alkyl) wherein x and w are independently 1, 2 or 3;
R^{A} is hydrogen or C₁-C₃ alkyl optionally substituted with one or more fluorine substituents; and
Alk³ and Alk⁴ are divalent C₁-C₃ alkylene or C₂-C₃ alkenylene radicals, each optionally substituted by one or two substituents selected from fluoro, chloro, C₁-C₃alkyl optionally substituted with one or more fluorine substituents, C₁-C₃alkoxy optionally substituted with one or more fluorine substituents.

4. A compound as claimed in claim 1 wherein R₁ and SI are independently selected from (a) hydrogen, methoxy, ethoxy, methylthio or ethylthio; (b) a group of formula —X¹-Alk⁵-(CO)_{w}NR_{C}R^{D} wherein w is 0 or 1, X¹ is —O— or —S—, Alk⁵ is a straight or branched chain C₁-C₃alkylene radical, R^{C} is C₁-C₃alkyl and R^{D} is C₁-C₃alkyl or hydroxyl(C₁-C₃alkyl)-; and (c) a group of formula —X¹-Alk⁵-Ar wherein X¹ is —O— or —S—, Alk⁵ is a straight or branched chain C₁-C₃alkylene radical, and Ar is phenyl or a 5- or 6-membered heteroaryl ring wherein at least one hetero atom is nitrogen

5. A compound as claimed in claim 1 wherein R₁ is methoxy, ethoxy, methylthio or ethylthio.

6. A compound as claimed in claim 1 wherein R₂ is cyano (—CN).

7. A compound as claimed claim 2 wherein R₃ is in the ortho position and R₄ in the para position.

8. A compound as claimed in claim 2 wherein S₁ is in the meta position of the phenyl ring.

9. A compound as claimed in claim 1 wherein R₃ and/or R₄ is selected from fluoro, chloro, bromo and methyl.

10. A compound as claimed in claim 1 having formula (ID): 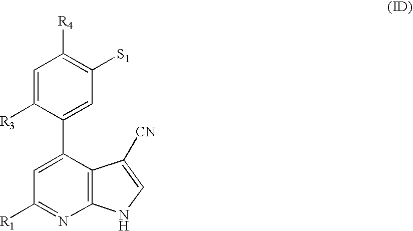
or a salt, N-oxide, hydrate, or solvate thereof, wherein:
R₁ and S₁ are independently selected from (a) hydrogen, methoxy, ethoxy, methylthio or ethylthio; (b) a group of formula —X¹-Alk⁵-(CO)_{w}NR^{C}R^{D} wherein w is 0 or 1, X¹ is —O— or —S—, Alk⁵ is a straight or branched chain C₁-C₃alkylene radical, R^{C} is C₁-C₃alkyl and R^{D} is C₁-C₃alkyl or hydroxyl(C₁-C₃alkyl)-; and (c) a group of formula —X¹-Alk⁵-Ar wherein X¹ is —O— or —S—, Alk⁵ is a straight or branched chain C₁-C₃alkylene radical, and Ar is phenyl or a 5- or 6-membered heteroaryl ring wherein at least one hetero atom is nitrogen; PROVIDED THAT R₁ and S₁ are not both hydrogen;
R₃ is fluoro, chloro, bromo or methyl; and
R₄ is fluoro, chloro, bromo methyl, ethyl, isopropyl, methoxy, or cyano.

11. A compound as claimed in claim 1 selected from the group consisting of:
4-(2,4-Dimethyl-phenyl)-1H-pyrrolo[2,3-b]pyridine-3-carbonitrile,
4-(2,4-Dichloro-phenyl)-1H-pyrrolo[2,3-b]pyridine-3-carbonitrile,
6-Chloro-4-(2,4-dichloro-phenyl)-1H-pyrrolo[2,3-b]pyridine-3-carbonitrile,
4-(2,4-Di chloro-5-methoxy-phenyl)-1H-pyrrolo[2,3-b]pyridine-3-carbonitrile,
6-Chloro-4-(2,4-dichloro-5-methoxy-phenyl)-1H-pyrrolo[2,3-b]pyridine-3-carbonitrile,
3-Bromo-4-(2,4-dimethyl-phenyl)-1H-pyrrolo[2,3-b]pyridine-3-carbonitrile,
4-(2,4-Dimethyl-phenyl)-1H-pyrrolo[2,3-b]pyridine-3-carbaldehyde,
4-(2,4-Difluorol-phenyl)-1H-pyrrolo[2,3-b]pyridine-3-carbaldehyde,
6-(2-Diethylamino-ethoxy)-4-phenyl-1H-pyrrolo[2,3-b]pyridine-3-carbonitrile,
4-(2,4-Dichloro-phenyl)-6-(2-diethylamino-ethoxy)-1H-pyrrolo[2,3-b]pyridine-3-carbonitrile,
4-(2,4-Dichloro-phenyl)-6-(3-piperidin-1-yl-propoxy)-1H-pyrrolo[2,3-b]pyridine-3-carbonitrile,
4-(3-Hydroxy-phenyl)-1H-pyrrolo[2,3-b]pyridine-3-carbonitrile,
4-[3-(2-Diethylamino-ethoxy)-phenyl]-1H-pyrrolo[2,3-b]pyridine-3-carbonitrile,
6-Chloro-4-(3-hydroxy-phenyl)-1H-pyrrolo[2,3-b]pyridine-3-carbonitrile, and
6-methoxy-4-(-phenyl)-1H-pyrrolo[2,3-b]pyridine-3-carbonitrile.

12. A pharmaceutical or veterinary composition comprising a compound as claimed in claim 1, together with one or more pharmaceutically or veterinarily acceptable carriers and/or excipients.

13. (canceled)

14. A method of treatment of diseases which are responsive to inhibition of HSP90 activity in mammals, which method comprises administering to the mammal an amount of a compound as claimed in claim 1 effective to inhibit said HSP90 activity.

15. The method as claimed in claim 14 for immunosuppression or the treatment of viral disease, inflammatory diseases such as rheumatoid arthritis, asthma, multiple sclerosis, Type I diabetes, lupus, psoriasis and inflammatory bowel disease; cystic fibrosis angiogenesis-related disease such as diabetic retinopathy, haemangiomas, and endometriosis; or for protection of normal cells against chemotherapy-induced toxicity; or diseases where failure to undergo apoptosis is an underlying factor; or protection from hypoxia-ischemic injury due to elevation of Hsp70 in the heart and brain; scrapie/CJD, Huntingdon's or Alzheimer's disease.

16. The method as claimed in claim 14, for the treatment of cancer.
